(19) United States
(12) Patent Application Publication
(10) Pub. No.: US 2005/0048073 A1
(43) Pub. Date: Mar. 3, 2005

(54) Methods of determining west nile virus epitopes and method of using the same

(76) Inventors: Anne S. De Groot, Providence, RI (US); William Martin, Cumberland, RI (US)

Correspondence Address: Elrifi, Ivor R.;Mintz Levin Cohn Ferris Glovsky and Popeo, One Financial Center, Boston, MA, 02111, US

(21) Appl. No.: 10/468,950

(22) PCT Filed: Feb. 28, 2002
(86) PCT No.: PCT/US02/06575

Related U.S. Application Data
(60) Provisional application No. 60/272,132, filed on Feb. 28, 2001.

(51) Int. Cl. C12Q 1/70; A61K 39/12

(52) U.S. Cl. 424/186.1; 435/5

(57) ABSTRACT

Vaccines containing one or more West Nile Virus (WNV) vaccine candidate peptides in an immunologically acceptable excipient are disclosed. Also provided are recombinant WNV vaccine candidate peptides, wherein the peptide is expressed from a recombinant polynucleotide such as a naked DNA vaccine. Additionally, methods for inducing anti-WNV immune responses in a mammalian subject are also disclosed.

### TECHNICAL FIELD OF THE INVENTION

This invention relates generally to the fields of epidemiology, immnunology, and molecular biology.

### BACKGROUND

West Nile virus (WNV) is the cause of a potentially fatal form of viral encephalitis that suddenly emerged in the New York City area during 1999. The virus is a member of the flavivirus family. Other members of the same family include St. Louis Encephalitis, Japanese Encephalitis Virus (JEV), Hepatitis C and Dengue. WNV is commonly found in West Asia, Africa, and the Middle East but was not reported in the Americas until 1999. (Lanciotti et al., Science 286:2333-37 (1999); Wright et al., Aust. J. Exp. Biol. Med. Scie. 61(Pt. 6):641-53 (1983)). The source of the introduction of the virus to New York City is unknown. Introduction by an infected host (e.g. human or bird), by an infected vector (e.g. mosquito), or by bio-terrorists are potential sources of WNV listed by the United States Centers for Disease Control.

Surveillance data reported to the CDC have indicated intensified transmission and geographic expansion of the West Nile Virus (NY99) outbreak in the northeastern United States during the last two years. Twelve states and the District of Columbia reported WNV epizootic activity in 2000, a significant increase over the four states reporting activity in 1999. West Nile Virus is expected to continue to spread along the East Coast of the United States in 2001 and years thereafter due to over-wintering of mosquitoes and avian migratory patterns. (Andersen et al., Science 286:2331-33 (1999); Rappole et al., Emerging Infectious Diseases 6(4):319-28 (2000)). Concern about the dissemination of WNV in the United States is supported by knowledge of current endemics and epidemics in other regions of the world. The largest African epidemic, with approximately 3,000 clinical cases, occurred in South Africa after heavy rains in 1974. Other outbreaks have been observed in the former Soviet Republic, Central African Republic, Kisangani in the Democratic Republic of Congo (former Zaire), Egypt, Ethiopia, India, Israel, Madagascar, Nigeria, Pakistan, Senegal, Sudan, and quite a few European countries.

The West Nile NY99 virus that was eventually associated with the New York 1999 outbreak appears to have been circulating in Israel since 1997. Other close relatives to the West Nile NY99 virus were isolated in Italy (1998), Morocco (1996), Romania (1996), and Africa (1989, 1993, 1998).

The epitope-driven vaccine concept is an attractive one that is being successfully pursued in a number of laboratories. See, e.g., Hanke et al., Vaccine 16:426 (1998); Ling-Ling et al., J. Virology 71:2292-302 (1997); Nardin et al., Immunol. 166(1):481-89 (2001).

### SUMMARY OF THE INVENTION

The goal of this project was to demonstrate the utility of a bioinformatics/computational immunology approach for the rapid selection of epitope reagents that would permit the evaluation of cell-mediated responses in the immunopathogenesis of West Nile Virus (WNV).

In one embodiment, the invention is concerned with the development of diagnostic reagents such as tetramers and preventive or therapeutic vaccines. (Altman et al., Science 274(94):6 (1996)).

In one aspect, the invention includes a vaccine that includes one or more West Nile Virus (WNV) candidate peptides disclosed in SEQ ID NOs: 1-95 and is in an immunologically acceptable excipient. For example, the vaccine could contain a combination of 2, 3,4, 5, 6, etc., WNV peptides disclosed in SEQ ID NOs:1-95.

In one embodiment, the invention includes a vaccine where the length of one or more WNV candidate peptides is between 8 amino acids and 10 amino acids in length.

In another embodiment, the invention includes a vaccine where one or more WNV candidate peptides have amino acid sequences from the group disclosed in SEQ ID NOs: 5, 8, 9, 13, 15, and 17-20.

The invention also includes a vaccine containing one or more WNV candidate peptides, where the peptide is complexed to a carrier protein. The carrier protein may be a recombinant fusion protein. Additionally, the excipient may be an adjuvant.

In another aspect, the invention includes one or more recombinant WNV candidate peptide where the peptides contain an amino acid sequence from the group disclosed in SEQ ID NOs: 5, 8, 9, 13, 15, and 17-20 and is expressed from a recombinant polynucleotide. In one embodiment, the recombinant polynucleotide is a naked DNA vaccine.

In another embodiment, the invention involves a method for inducing an anti-WNV immune response by administering a vaccine containing one or more WNV vaccine candidate peptides selected from the group consisting of SEQ ID NOs:1-95 and an immunologically acceptable excipient to a mammal. In a further embodiment, the induction of an anti-WNV immune response results in the raising of an anti-WNV antibody. In various embodiments, suitable mammals include, for example, humans, cows, pigs, horses, and dogs. Administration of the vaccine according to the invention may be orally, topically, parenterally, by viral infection, and/or intravascularly.

In another embodiment, the invention involves a method for inducing an anti-WNV immune response by administering a vaccine candidate peptide containing an amino acid sequence selected from the group consisting of SEQ ID NOs: 5, 8, 9, 13, 15, and 17-20, wherein the peptides are expressed from a recombinant polynucleotide. In a further embodiment, the induction of an anti-WNV immune response results in the raising of an anti-WNV antibody. In various embodiments, suitable mammals include, for example, humans, cows, pigs, horses, and dogs. Administration of the vaccine according to the invention may be orally, topically, parenterally, by viral infection, and/or intravascularly.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1a shows all 3,423 peptides obtained by parsing the West Nile Virus genome were scored using EpiMatrix and the EpiMatrix motif for HLA B*07 (this matrix motif provides equivalent scores for most of the HLA B*07 subtypes). HLA B*07 is a class I major histocompatability complex (“MHC”) antigen. Based on previous experience, most peptides (approximately 80%) scoring above an estimated binding probability (“EBP”) of 7 for this particular matrix motif are considered likely to bind to HLA B*07 in T2 B-7 assays. Twenty WNV peptides scoring between 20 and 50 were selected for the present study.

FIG. 1b shows EpiMatrix HLA B*07 score distributions for a random set of 10,000 peptides, 20 peptides selected for the WNV genome, and a set of known HLA B*07 ligands. The log of EBP for all three sets (random, known binders, WNV selection) fell in the range−5 to 5. The graph shows a frequency analysis of the data, graphing the proportion of peptides (over the total number for each set) falling within scores from −5 to 5.

FIG. 2 is a plot showing the distribution of EpiMatrix (“EMX”) scores along the length of the entire WNV genome.

### DETAILED DESCRIPTION OF THE INVENTION

The NY 1999 WNV sequence was obtained from Genbank (Accession No. AF196835). (Altschul et al., Nucleic Acids Res. 25:3389-402 (1997)). The WNV genome is 11,000 nucleotides long. The following proteins have been tentatively identified: major envelope glycoprotein E; membrane (envelope) protein M; non-structural proteins NS1, NS2A, NS3, NS4A; and RNA directed polymerase (non-structural protein 5). The CDC has completely sequenced the West Nile NY99 virus originally obtained from a Chilean flamingo, a West Nile NY99 equine isolate, the Italy 1998 virus, the Romania 1996 virus and the prototype Eg101, virus. While the latter viruses are closely related to West Nile NY99, they are neither identical to each other nor to West Nile NY99.

A virus isolated in Israel, “Israel 1998”, appears to be virtually identical to the West Nile NY99. Completion of the genome sequence of the Israel 1998 West Nile virus is being pursued at the Institute Pasteur, France.

Immune response to WNV has not been well delineated. CD4 T helper responses have been identified to related viruses (e.g. JEV, Dengue) and are felt to be essential components of protective immune response. Also, mobilization of dendritic cells and antigen presentation by Langerhans cells found in the dermis to T cells found in the lymphoid follicles may be involved in the development of immune responses to WNV. (Johnston et al., J. Invest. Dermatol. 114(3):560-68 (2000)).

Some CD4 T cells can be identified that respond to epitopes within JEV that are identical, or nearly identical, with sequences contained in WNV. For example, T cell clones have been derived from a human subject who experienced dengue illness following immunization with a live experimental dengue virus type 3 (DEN3 vaccine). The NS3 protein was immunodominant in the CD4+ T-cell response of this subject. The epitopes of four Dengue NS3-specific T-cell clones were analyzed; one of the four recognized peptide epitopes derived from WNV as well as JEV. These epitopes were fine-mapped. The smallest synthetic peptide recognized by these T cell clones was a nine amino acid peptide containing amino acids 146 to 154 of dengue-4 NS3. These results confirmed immunologic cross-reactivity between JEV and WNV.

Other researchers have focused on the effect of WNV and other flavivirus family members on endothelial cells. Both ICAM-1 and MHC class I and II expression are upregulated in human endothelial cells in the first 72 hours of infection. Langerhans cells (LC) in the epidermis may play a role in the upregulation of immune response to the virus, processing antigen, and presenting it to T cells. Furthermore, researchers have postulated that LC may migrate to local lymph nodes following cutaneous infection with WNV.

Mobilization of dendritic cells and antigen presentation by these cells to T cells in the lymphoid follicles may therefore be involved in the development of immune responses to WNV.

In support of this hypothesis, cytotoxic T cell responses (restricted by class I MHC) and T helper responses (restricted by class II MHC) appear to be critical components of human immune response to the other members of the flavivirus family. (Lobigs et al., Virology 202(1):195-201 (1994); (Murali-Krishna et al., J. Gen. Virol. 75(Pt. 4):799-807 (1994)). Development of cell-mediated immunity to WNV may be a critically important barrier to infection of the central nervous system. Reagents that permit the evaluation of cell-mediated responses may permit researchers to better understand the immunopathogenesis of WNV, to develop diagnostic reagents such as tetramers, and to develop preventive or therapeutic vaccines.

“MHC tetramers” are a new tool that may make field-based screening for immune responses to emerging viruses an important epidemiological tool. Tetramers were first developed a few years ago by Altman et al., Science 274(94):6 (1996). These specialized constructs bear four MHC molecules complexed with beta 2 microglobulin and a specific pathogen-derived peptide ligand. These novel reagents can bind directly to T cells that recognize the MHC-peptide complex. They can be used for direct ex vivo analysis of the frequency and phenotypes of antigen-specific T cells by flow cytometry. The tetramer staining assay relies only upon the interaction between the tetramer reagent and T cell receptor clusters (and possibly co-receptors) on the surface of T cells. The assay reduces to an absolute minimum the in vitro manipulation of the sample before detection of the antigen-specific population.

The incubation period in humans (i.e., time from infection to onset of disease symptoms) for WNV encephalitis is usually 5 to 15 days. Antibodies are detectable within three to seven days. In contrast, based on recent tetramer-staining studies, WNV T cell responses should be detectable within two to three days of infection. The initial CTL response to acute infection with a virus, as measured using tetramer technology, is phenomenal. For example, during the acute immune response to lymphocytic choriomeningitis virus (LCMV) in BALB/c mice, 55% of all CD8+ splenocytes are stained with a LCMV specific tetramer. The method is extremely robust, and can detect antigen-specific populations at frequencies as low as 1:5,000 CD8+ T cells (or approximately 1:50,000 PBMC).

As little as 2 cc of blood can be drawn (containing approximately 2 million peripheral blood mono-nuclear cells, PBMC), mixed with tetramer reagents, and analyzed by fluorescence-activated cell sorting (FACS). No incubation is required, and the assay does not require a priori assumptions of the class of functional responses (e.g. cytokine profiles), and is therefore likely to provide the most complete method for detection of the magnitude of an antigen-specific response. Results can be available within 60 minutes of drawing a blood sample without any additional processing (other than mixing the tetramer reagent with the blood sample). It is conceivable that companies holding the patent for tetramer diagnostics (Beckman Coulter, for example) will begin to develop low cost FACS machines for use in the field.

There is no specific vaccine or anti-viral treatment available for WNV infection. Patients who develop symptoms of WNV encephalitis can be managed with supportive intensive care. Not all patients with severe neurological disease due to WNV recover. It is likely that a CTL response will be one critical component of immune response against WNV. The development of a preventive or therapeutic vaccine against this public health threat would be greatly expedited if the correlates of immune response were determined and if those correlates could be rapidly incorporated into a vaccine. Epitopes defined using methods such as the one described here may also be useful for evaluating immune response to WNV and developing vaccines.

West Nile Virus is expected to continue to spread along the East Coast of the United States in the future due to over-wintering of mosquitoes and avian migratory patterns. Although the CDC has made recommendations that will reduce the incidence of WNV transmission in populated areas, once the virus has become established it is unlikely to be eradicated. Tools that will enhance epidemiological surveillance, case detection, diagnosis, research on the immunopathogenesis of WNV are likely to be of great interest to physicians, public health officials, and the lay public during vectored transmission seasons (April to November). A bioinformatics/computational immunology approach to epitope discovery, such as the one illustrated here, will make significant contributions to the development of new research and diagnostic reagents for West Nile Virus and other emerging infectious diseases.

Once the epitope candidates selected using this method are confirmed in CTL assays, they may be useful for (1) screening exposed individuals, (2) investigating the immuno-pathogenesis of WNV disease in humans, (3) as components of diagnostic kits developed for the surveillance effort, and/or (4) eventually, as a tool for measuring WNV vaccine-related immune responses. Confirmation of T cell response to the peptides will depend on availability of peripheral blood cells from West Nile-infected patients during the next transmission season. Additional peptides also need to be defined and screened for binding to other HLA alleles, in order to broaden the MHC specificity of the diagnostic reagent or immunopathogenesis tools developed using this approach.

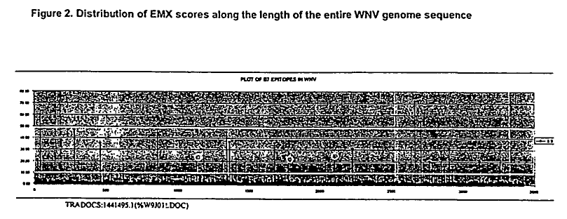

Peptides selected by the EpiMatrix approach were not confined to any particular protein, as illustrated in FIG. 2. The final set of peptides included four from NS-1, four from NS-2A, five from NS-3, one from NS-4A, five from NS-5, one from envelope protein E, and one from membrane protein M.

A summary of WNV peptides according to the invention is given below in Table 1. Each of the 95 peptide sequences identified is shown in Column 1. Column 2 shows the sequence of each peptide. Column 4 shows the location of the amino acid start. The EBP for each peptide is shown in Column 4. Column 5 shows the “cover” value for each peptide.

TABLE 1

| | | | | | |
|---|---|---|---|---|---|
| SEQ ID | | | | | |
| NO. | Dataset | AA Sequence | AA Start | E.B.P. | Cover |

| | | | | | |
|---|---|---|---|---|---|
| 1 | WNB7 001 | RPSECCDTLL | 2663 | 72.10 | 10.20 |
| 2 | WNB7 002 | GPIRFVLALL | 42 | 59.88 | 19.22 |
| 3 | WNB7 003 | GPREFCVKVL | 2703 | 54.68 | 23.89 |
| 4 | WNB7 004 | AVKDELNTLL | 861 | 47.77 | 30.50 |
| 5 | WNB7 005 | APAYSFNCLG | 286 | 47.03 | 31.21 |
| 6 | WNB7 006 | AAKKKGASLL | 1337 | 45.30 | 33.00 |
| 7 | WNB7 007 | NPMILAAGLI | 1357 | 36.60 | 42.86 |
| 8 | WNB7 008 | IPAGFEPEML | 1680 | 36.02 | 43.64 |
| 9 | WNB7 009 | TPAAPSYTLK | 460 | 35.68 | 44.04 |
| 10 | WNB7 010 | VPCRGQDELV | 3259 | 32.57 | 48.01 |
| 11 | WNB7 011 | GPGHEEPQLV | 2635 | 32.35 | 48.40 |
| 12 | WNB7 012 | AGMLLLSLLL | 2229 | 31.17 | 50.40 |
| 13 | WNB7 013 | EPPEGVKYVL | 2895 | 31.07 | 50.40 |
| 14 | WNB7 014 | MPAILIALLV | 1177 | 30.23 | 51.60 |
| 15 | WNB7 015 | KPTGSASSLV | 2842 | 28.79 | 53.59 |
| 16 | WNB7 016 | IPTAAGKNLC | 148 | 25.56 | 58.32 |
| 17 | WNB7 017 | RPRWIDARVY | 2098 | 23.99 | 60.64 |
| 18 | WNB7 018 | VPGTKIAGML | 2223 | 23.10 | 62.17 |
| 19 | WNB7 019 | RPQRHDEKTL | 1127 | 22.49 | 62.93 |
| 20 | WNB7 020 | SPHRVPNYNL | 1777 | 22.40 | 63.31 |
| 21 | WNB7 021 | IPMTIAGLMF | 1405 | 22.32 | 63.31 |
| 22 | WNB7 022 | MPRVLSLIGL | 21 | 21.22 | 65.17 |
| 23 | WNB7 023 | RPAADGRTVM | 3112 | 20.89 | 65.54 |
| 24 | WNB7 024 | TPGLRCLNLD | 1306 | 20.73 | 65.91 |
| 25 | WNB7 025 | SVNMTSQVLL | 2760 | 20.09 | 67.00 |
| 26 | WNB7 026 | EERKNFLELL | 2034 | 19.15 | 68.44 |
| 27 | WNB7 027 | GPQYEEDVNL | 2779 | 19.15 | 68.44 |
| 28 | WNB7 028 | APWKIWMLRM | 1467 | 18.18 | 70.19 |
| 29 | WNB7 029 | NARDRSIALT | 765 | 17.03 | 72.24 |
| 30 | WNB7 030 | VPISSVASLN | 628 | 16.68 | 72.91 |
| 31 | WNB7 031 | KPWDTITNVT | 2860 | 15.94 | 73.89 |
| 32 | WNB7 032 | LPDALQTIAL | 2171 | 15.81 | 74.22 |
| 33 | WNB7 033 | RPRMCSREEF | 2920 | 15.81 | 74.22 |
| 34 | WNB7 034 | TVWRNRETLM | 521 | 15.35 | 75.17 |
| 35 | WNB7 035 | LIMKDGRTLV | 3249 | 15.10 | 75.49 |
| 36 | WNB7 036 | KSYAQMWLLL | 3289 | 13.64 | 78.23 |
| 37 | WNB7 037 | FVDVGVSALL | 2361 | 13.46 | 78.52 |
| 38 | WNB7 038 | GPRTNTILED | 2072 | 12.90 | 79.67 |
| 39 | WNB7 039 | GTRAVGKPLL | 2791 | 12.57 | 80.23 |
| 40 | WNB7 040 | VPREHNGNEI | 1753 | 12.51 | 80.23 |
| 41 | WNB7 041 | GAPWKIWMLR | 1466 | 12.30 | 80.51 |
| 42 | WNB7 042 | IAGMLLLSLL | 2228 | 12.14 | 81.06 |
| 43 | WNB7 043 | MPKVIEKMEL | 2716 | 11.93 | 81.33 |
| 44 | WNB7 044 | EPVGKVIDLG | 2600 | 11.88 | 81.33 |
| 45 | WNB7 045 | RWFVVLLLL | 275 | 11.82 | 81.59 |
| 46 | WNB7 046 | AYHDARQILL | 1260 | 11.82 | 81.59 |
| 47 | WNB7 047 | APKRLTATTE | 891 | 11.77 | 81.59 |
| 48 | WNB7 048 | EPRSGIDTNA | 481 | 11.67 | 81.86 |
| 49 | WNB7 049 | GGRAHRMALE | 2160 | 11.57 | 82.12 |
| 50 | WNB7 050 | EPPFGDSYIV | 666 | 11.22 | 82.64 |
| 51 | WNB7 051 | MIDPFQLGLL | 1148 | 10.98 | 83.15 |
| 52 | WNB7 052 | ILRNPGYALV | 251 | 10.88 | 83.40 |
| 53 | WNB7 053 | AAPSYTLKLG | 462 | 10.88 | 83.40 |
| 54 | WNB7 054 | TPWAILPSVV | 1485 | 10.74 | 83.65 |
| 55 | WNB7 055 | KPLDDRFATS | 3201 | 10.59 | 83.89 |
| 56 | WNB7 056 | AKSYAQMWLL | 3288 | 10.09 | 84.85 |
| 57 | WNB7 057 | AIPMTIAGLM | 1404 | 9.91 | 85.08 |
| 58 | WNB7 058 | GPCKVPISSV | 624 | 9.87 | 85.31 |
| 59 | WNB7 059 | KGPKVRTWLF | 3172 | 9.87 | 85.31 |
| 60 | WNB7 060 | APELANNTFV | 916 | 9.56 | 85.77 |
| 61 | WNB7 061 | GARFLEFEAL | 3010 | 9.52 | 85.77 |
| 62 | WNB7 062 | HSRRSRRSLT | 209 | 9.35 | 86.21 |
| 63 | WNB7 063 | TPADTGHGTV | 604 | 9.35 | 86.21 |
| 64 | WNB7 064 | NVVVPLLALL | 1296 | 9.27 | 86.43 |
| 65 | WNB7 065 | GPGKSRAVNM | 7 | 9.06 | 86.65 |
| 66 | WNB7 066 | EPHATKQSVI | 534 | 9.06 | 86.65 |
| 67 | WNB7 067 | GPWDEGRVEI | 1057 | 8.98 | 86.86 |
| 68 | WNB7 068 | WPATEVMTAV | 1376 | 8.82 | 87.29 |
| 69 | WNB7 069 | MLRKKQITVL | 1688 | 8.74 | 87.29 |
| 70 | WNB7 070 | AAAKKKGASL | 1336 | 8.66 | 87.49 |
| 71 | WNB7 071 | TVTVTAATLL | 2382 | 8.62 | 87.49 |
| 72 | WNB7 072 | AGCWGQVTLT | 2373 | 8.47 | 87.90 |
| 73 | WNB7 073 | VPNYNFLVMD | 1781 | 8.32 | 88.10 |
| 74 | WNB7 074 | AVFLICVMTL | 2258 | 8.24 | 88.30 |
| 75 | WNB7 075 | KPTIDVKMMN | 328 | 8.20 | 88.30 |
| 76 | WNB7 076 | GMSWITQGLL | 746 | 8.09 | 88.49 |
| 77 | WNB7 077 | KCRVKMEKLQ | 577 | 8.02 | 88.69 |
| 78 | WNB7 078 | AVGGVLLFLS | 777 | 7.95 | 88.88 |
| 79 | WNB7 079 | LAREKRPRMC | 2915 | 7.91 | 88.88 |
| 80 | WNB7 080 | EPGKNVKNVQ | 1606 | 7.73 | 89.25 |
| 81 | WNB7 081 | ADMIDPFQLG | 1146 | 7.70 | 89.25 |
| 82 | WNB7 082 | RGMPRVLSLI | 19 | 7.66 | 89.44 |
| 83 | WNB7 083 | FCSNHFTELI | 3241 | 7.66 | 89.44 |
| 84 | WNB7 084 | VYRIMTRGLL | 1527 | 7.52 | 89.62 |
| 85 | WNB7 085 | DPFQLGLLVV | 1150 | 7.46 | 89.80 |
| 86 | WNB7 086 | TAIAPTRAVL | 57 | 7.42 | 89.80 |
| 87 | WNB7 087 | LKRYEDTTLV | 3419 | 7.39 | 89.97 |
| 88 | WNB7 088 | SMPAILIALL | 1176 | 7.32 | 89.97 |
| 89 | WNB7 089 | LVNGVVRLLS | 2850 | 7.32 | 89.97 |
| 90 | WNB7 090 | LVAAVIGWML | 259 | 7.29 | 90.15 |
| 91 | WNB7 091 | LGMSNRDFLE | 294 | 7.29 | 90.15 |
| 92 | WNB7 092 | RVKMEKLLQLK | 579 | 7.29 | 90.15 |
| 93 | WNB7 093 | GPRSNHNRRP | 1040 | 7.25 | 90.15 |
| 94 | WNB7 094 | AIAPTRAVLD | 58 | 7.12 | 90.49 |
| 95 | WNB7 095 | MPNGSYISAI | 1661 | 7.02 | 90.66 |

EBPs for the WNV peptides ranged from >20% (highly likely to bind) to <1% (very unlikely to bind). Peptides with EpiMatrix EBP scores in the range of 7 to 50 are more likely to bind to MHC and stimulate T cells in vitro. See Jin et al., AIDS Res Hum Retroviruses 16:67-76 (2000). Peptides with an EBP score 50 are less likely to be immunogenic. However, they may bind to B7 in vitro. See Jin et al., supra; DeGroot et al., Vaccine 2000 (in press). [HAS THIS PAPER PUBLISHED ALREADY?]

Triplicate wells of peptide at 10, 20, 200 ug/ml were evaluated in each of the T2 B7 binding assays; each assay was repeated 4 times. Table 2 provides information on the MFI for the peptide at 200 ug/ml, the fold increase over background for the peptide at 200 ug/ml, and a summary of binding results in each of the assays. In the summary “binding results” column, “0/3” signifies none of the wells at any of the three concentrations gave a positive result (in one assay plate), “×4” signifies that this result was obtained in each of four assays; “×3”” signifies that this result was obtained in each of three assays, and so on. The “binding score” column provides the numerical sum of all the positive assays in the binding summary column. For example, summing the results for peptide 0005: 3/3×1, 2/3×2, 1/3×1=(3×1)+(2×2)+(1×1)=8.

TABLE 2

WNV T2B7 binding assay results

| | | | |
|---|---|---|---|
| | Avg. | | |
| SEQ | Fold | Avg. | Binding |

| | | | | | | | | |
|---|---|---|---|---|---|---|---|---|
| ID | | | | Inc. @ | MFI¹ | results (total | Binding | |
| NO: | Peptide | AA Sequence | EBP | 200 | 200 | of four assays | number | strength |

| | | | | | | | | | |
|---|---|---|---|---|---|---|---|---|---|
| 4 | WNVB7 | AVKDELNTLL | 47.77 | 1.0 | 842.5 | 0/3(×4) | 0 | non-binder | n |
| | 0004 | | | | | | | | |
| 5 | WNVB7 | APAYSFNCLG | 47.03 | 1.2 | 743.5 | 3/3(×1), | 8 | moderate | y |
| | 0005 | | | | | 2/3(×2), | | | |
| | | | | | | 1/3(×1) | | | |
| 6 | WNVB7 | AAKKKGASLL | 45.30 | 1.2 | 708.1 | 1/3 × 4 | 4 | weak | y |
| | 0006 | | | | | | | | |
| 7 | WNVB7 | NPMILAAGLI | 36.60 | 1.1 | 944.6 | 1/3(×2) | 2 | non-binder | n |
| | 0007 | | | | | | | | |
| 8 | WNVB7 | IPAGFEPEML | 36.02 | 1.9 | 1207.4 | 3/3 × 3, | 10 | strong | y |
| | 0008 | | | | | 1/3 × 1 | | | |
| 9 | WNVB7 | TPAAPSYTLK | 35.68 | 1.5 | 954.5 | 3/3(×1), | 8 | moderate | y |
| | 0009 | | | | | 2/3(×2), | | | |
| | | | | | | 1/3(×1) | | | |
| 10 | WNVB7 | VPCRGQDELV | 32.57 | 1.0 | 658.6 | 2/3(×2) | 4 | weak | y |
| | 0010 | | | | | | | | |
| 11 | WNVB7 | GPGHEEPQLV | 32.35 | 1.0 | 848.9 | 2/3(×1) | 2 | non-binder | n |
| | 0011 | | | | | | | | |
| 12 | WNVB7 | AGMLLLSLLL | 31.17 | — | — | — | — | — | |
| | 0012 | | | | | | | | |
| 13 | WNVB7 | EPPEGVKYVL | 31.07 | 1.1 | 681.7 | 3/3(×1), | 6 | moderate | y |
| | 0013 | | | | | 2/3(×1), | | | |
| | | | | | | 1/3(×1) | | | |
| 14 | WNVB7 | MPAILIALLV | 30.23 | — | — | — | — | — | |
| | 0014 | | | | | | | | |
| 15 | WNVB7 | KPTGSASSLV | 28.79 | 1.7 | 1070.2 | 2/3(×2), | 6 | moderate | y |
| | 0015 | | | | | 1/3(×2) | | | |
| 17 | WNVB7 | RPRWIDARVY | 23.99 | 1.9 | 1714.0 | 3/3(×3), | 11 | strong | y |
| | 0017 | | | | | 2/3(×1) | | | |
| 18 | WNVB7 | VPGTKIAGML | 23.10 | 1.7 | 1088.4 | 3/3 × 1, | 6 | moderate | y |
| | 0018 | | | | | 2/3 × 1, | | | |
| | | | | | | 1/3 × 2 | | | |
| 19 | WNVB7 | RPQRHDEKTL | 22.49 | 2.8 | 1644.6 | 3/3 × 3, | 11 | strong | y |
| | 0019 | | | | | 2/3 × 1 | | | |
| 20 | WNVB7 | SPHRVPNYNL | 22.40 | 2.5 | 1607.9 | 3/3(×4) | 12 | strong | y |
| | 0020 | | | | | | | | |
| 21 | WNVB7 | IPMTIAGLMF | 22.32 | — | — | — | — | — | |
| | 0021 | | | | | | | | |
| 23 | WNVB7 | RPAADGRTVM | 20.89 | 1.5 | 979.1 | 3/3 × 4 | 12 | strong | y |
| | 0023 | | | | | | | | |
| 24 | WNVB7 | TPGLRCLNLD | 20.73 | 1.0 | 849.6 | 1/3(×2) | 2 | non-binder | n |
| | 0024 | | | | | | | | |
| 25 | WNVB7 | SVNMTSQVLL | 20.09 | — | — | — | — | — | |
| | 0025 | | | | | | | | |
| 96 | WNVB7 | PEDIDCWCTK | 0.00 | 1.1 | 990.3 | 1/3(×1) | 1 | non-binder | n |
| | 3399 | | | | | | | | |
| 97 | WNVB7 | PETPQGLAKI | 0.00 | 1.0 | 588.9 | 0/3 × 4 | 0 | non-binder | n |
| | 3403 | | | | | | | | |
| 98 | WNVB7 | PFPESNSPIS | 0.00 | 1.0 | 626.7 | 1/3(×1) | 1 | non-binder | n |
| | 3411 | | | | | | | | |
| 99 | WNVB7 | PRTNTILEDN | 0.00 | 0.9 | 778.6 | 0/3(×4) | 0 | non-binder | n |
| | 3415 | | | | | | | | |
| 100 | HIV-1 B7 | GPGHKARVLA | GPGH | 2.2 | 1423.8 | 2/3 × 2, | 8 | moderate | 1 |
| | 1291 | | KARV | | | 3/3 × 1, | | | 2 |
| | | | LA | | | 1/3 × 1 | | | |
| | | | | 1.5 | 1370.9 | 3/3(×2), | 9 | moderate | 4 |
| | | | | | | 2/3(×1), | | | |
| | | | | | | 1/3(×1) | | | |
| | | | | 1.6 | 1003.9 | 3/3(×2), | 8 | moderate | 1 |
| | | | | | | 2/3(×1) | | | 6 |

¹MFI = mean fluorescence index and T2B7 binding assay results for each of the peptides.

Twelve of the sixteen WNV peptides selected for higher likelihood of binding to B7 and tested in vitro demonstrated consistent binding in the four replicate assays. Of these peptides, five (0008, 0017, 0019, 0020, and 0023) stabilized HLA B7 on the surface of T2B7 cells at more than two concentrations in all four replicate assays, receiving a total binding score of 10, 11, 11, 12, and 12, respectively (Table 2). Five WNV peptides (0005, 0009, 0013, 0015, 0018) stabilized HLA B*07 to a moderate degree (receiving binding scores of 6 to 8). Two WNV peptides (0006, 0010) were weak binders (binding score of 4) and three did not bind (binding score of 2).

Table 3a shows the selected WNV peptides and their respective EpiMatrix scores. Table 3b shows some additional peptides that were not tested and provides reasons why such testing was not done.

TABLES 3a and 3b

Selected WNV peptides and their
EpiMatrix scores

| | | | | | |
|---|---|---|---|---|---|
| SEQ | peptide | | | | EBP |
| ID | number (B*07 | | | AA | (EMX |
| NO: | rank) | Source | Sequence | start | score) |

| | | | | | |
|---|---|---|---|---|---|
| 4 | WNB7 0004 | NS-1 | AVKDELNTLL | 861 | 48 |
| 5 | WNB7 0005 | mpM | APAYSFNCLG | 286 | 47 |
| 6 | WNB7 0006 | NS-2A | AAKKKGASLL | 1337 | 45 |
| 7 | WNB7 0007 | NS-2A | NPMILAAGLI | 1357 | 37 |
| 8 | WNB7 0008 | NS-3 | IPAGFELEML | 1680 | 36 |
| 9 | WNB7 0009 | env gp E | TPAAPSYTLK | 460 | 36 |
| 10 | WNB7 0010 | NS-5 | VPCRGQDELV | 3259 | 33 |
| 11 | WNB7 0011 | NS-5 | GPGHEEPQLV | 2635 | 32 |
| 12 | WNB7 0012 | NS-4A | AGMLLLSLLL | 2229 | 31 |
| 13 | WNB7 0013 | NS-5 | EPPEGVKYVL | 2895 | 31 |
| 14 | WNB7 0014 | NS-2A | MPAILLALLV | 1177 | 30 |
| 15 | WNB7 0015 | NS-5 | KPTGSASSLV | 2842 | 29 |
| 17 | WNB7 0017 | NS-3 | RPRWIDARVY | 2098 | 24 |
| 18 | WNB7 0018 | NS-4A | VPGTKIAGML | 2223 | 23 |
| 19 | WNB7 0019 | NS-1 | RPQRHDEKTL | 1127 | 22 |
| 20 | WNB7 0020 | NS-3 | SPHRVPNYNL | 1777 | 22 |
| 21 | WNB7 0021 | NS-2A | IPMTIAGLMF | 1405 | 22 |
| 23 | WNB7 0023 | NS-5 | RPAADGRTVM | 3112 | 21 |
| 24 | WNB7 0024 | NS-2A | TPGLRCLNLD | 1306 | 21 |
| 25 | WNB7 0025 | NS-5 | SVNMTSQVLL | 2760 | 20 |
| 96 | WNB7 3399 | pre-mpM | PEDIDCWCTK | 185 | 0 |
| 97 | WNB7 3403 | NS-1 | PETPQGLAKI | 827 | 0 |
| 101 | WNB7 3407 | NS-1 | PRSNHNRRPG | 1041 | 0 |
| 98 | WNB7 3411 | NS-3 | PFPESNSPIS | 1830 | 0 |
| 99 | WNB7 3415 | NS-3 | PRTNTILEDN | 2073 | 0 |

TABLE 3b

| | | | | | |
|---|---|---|---|---|---|
| SEQ | peptide | | | | EBP |
| ID | number | Reason for | | AA | (EMX |
| NO: | (B*07 rank) | not testing | Sequence | start | score) |

| | | | | | |
|---|---|---|---|---|---|
| 1 | WNB7 | EBP > 50 | RPSECCDTLL | 2663 | 72 |
| | 0001 | | | | |
| 2 | WNB7 | EBP > 50 | GPIRFVLALL | 42 | 60 |
| | 0002 | | | | |
| 3 | WNB7 | EBP > 50 | GPREFCVKVL | 2703 | 55 |
| | 0003 | | | | |
| 16 | WNB7 | not | IPTAAGKNLC | 148 | 26 |
| | 0016 | expressed | | | |
| 22 | WNB7 | not | MPRVLSLIGL | 21 | 21 |
| | 0022 | expressed | | | |

3393 additional peptides not tested (including 69 putative B*07 binders and 3353 “non-binders”)

The 25 peptides that were tested in vitro are shown in Table 3a. Twenty of the peptides were selected because they had an EpiMatrix (EMX) score (estimated binding probability, or EBP) between 7 and 50.

Peptides that were not tested although they may have fit the above criteria are listed in Table 3b. Three peptides received EMX scores above 50 (0001, 0002, 0003); these peptides were considered to be unlikely to be epitopes based on previous experience with HLA B*07 restricted HIV-1 epitopes. An additional two peptides were not selected, even though they were predicted to be binders (0016, 0022), because they fell in regions of the genome that were considered unlikely to be expressed, based on information provided by Genbank.

The control peptide, WC 1291, was tested in binding assays 3 times (for each set of peptides). The peptide bound moderately well, receiving a total binding score of eight to nine. Four WNV peptides selected for low EBP (3399, 3404, 3411, and 3415, all receiving EBPs of 0.0%) did not stabilize T2B7 to a significant degree, receiving binding scores of 0 to 1, reflecting only occasional stabilization of HLA B7 over background.

Peptides 0008 and 0019 were fairly unique, when compared to sequences of other viruses within the same flavivirus family and to sequences available in public databases. Peptide 0008, a strong binder, was 100% conserved in most strains of West Nile virus, and different by one amino acid from Kunjin virus (closely related). Peptide 0019, another strong binder, was 100% conserved in WNV and Kunjin virus and 80% conserved in JEV. Peptide 0017 was 100% conserved in all strains of WNV and Kunjin virus, 80% conserved in JEV and MVEV, and 90% conserved in some strains of Dengue. Peptide 0020, another strong binder, was conserved in West Nile and Kunjin (100%), JEV, MVEV, and Dengue (90%).

Of the five moderately well binding peptides (0005, 0009, 0013, 0015 and 0018), 0005 was 100% conserved across WNV, Kunjin, SLE and Sindbis virus. Thus, it is an interesting candidate for a vaccine against all members of this family of flaviviruses. Peptide 0009, in contrast, was only conserved in WNV and Kunjin. Peptide 0013, likewise, was conserved in WNV and Kunjin but less well in JEV (80%). WNV 0015 was conserved in WNV, Kunjin (100%), JEV (90%), MVEV (80%), SLEV (90%) and Dengue (80%). WNV 0018 was conserved in WNV, JEV (90%), MVEV (80%), and SLEV (90%). Of the two weak binding WNV peptides (0006,0010), 0006 was 90-100% conserved in WNV, Kunjin, JEV, and MVEV. In contrast, weak binder 0010 was highly conserved (as high as 90%) in a very wide range of viruses (West Nile, Kunjin, Japanese encephalitis, Murray Valley encephalitis, St. Louis encephalitis, Sindbis virus, Dengue, Koutango,Alfuy, Usutu, Ntaya, Saumarez Reef, Langat, Ilheus, Meaban, Yellow fever, Cacipacore, Russian Spring-Summer encephalitis, Neudoerfl, Louping ill, Tick-borne, Kadam, Royal Farm, Kyasanur forest disease, Edge Hill, Negishi, Karshi, Omsk hemorrhagic fever, Gadgets Gully, Powassan, Tyuleniy, Stratford, Kokobera, Rocio, Sitiawan, Sokuluk).

An additional 69 WNV peptides that might be expected to bind to HLA B*07 (based on score above 7) and to stimulate T cell responses (based on score below 50 and above 7) were not tested due to funding constraints and the pilot nature of this project. If the reminder of the WNV B7 peptides behave as predicted, we would expect that an additional 62 ligands to be identified (for a total of 71 out of the total 94 selected). Based on the HIV-1 results, it is expected that approximately 56 of the peptides (60% of the original 94) would be recognized by human CTL.

Peptides selected using the EpiMatrix HLA B*07 matrix method and included in this study did not always fit the conventional, anchor based format of P in position 2 and L or F in position 9. One weak binder, AAKKKGASLL (SEQ ID NO: 6), had little in common with published HLA B*07 motifs. Additional experiments were performed to determine whether peptides that are not selected for conformity for a given EpiMatrix scoring motif are likely to bind to that HLA molecule, in T2 binding assays. A set of 7 HIV-1 peptides that scored well on unrelated alleles (HLA A0201 and HLA A11) but poorly with respect to HLA B7 were tested. Only one of the seven peptides stabilized HLA B*07 on the surface of T2B7 cells.

Information derived from such peptides that deviate from the expected (selected ligands that do not bind, and selected non-binders that do bind) are integrated into EpiVax and TB/HIV Research Lab databases so as to improve the EpiMatrix scoring matrix model. HLA B*07 is found in 7.7% of Blacks, 8.7 % of caucasoids, 3% of orientals. Clearly there is a need to map additional epitopes for other alleles. EpiVax currently possesses information permitting the selection of putative MHC ligands for 30 class I alleles and 90 class II alleles. Mapping of additional epitopes for the WNV genome is currently underway.

Existing diagnostic tests for WNV are difficult to implement in the field, and due to the complexity of the tests and their interpretation, a great deal of inter-lab variability exists. Similarity between WNV and other members of the flavivirus family can also complicate the diagnosis of WNV infection. Patterns of antibody response are required to differentiate between WNV and related flaviviruses. Differentiation from Kunjin virus, counterpart or subtype of WNV found in Australia and Southeast Asia may be particularly difficult as most antibodies to Kunjin may be cross-reactive with WNV. Finally, the antibody tests (such as IgM capture EIA) that are available for detection of WNV infection rely on the availability of paired sera (two separate blood samples at two timepoints) to establish the diagnosis.

Plaque tests, using vero cells, are also used to detect live virus and may be most useful to confirm mosquito infection with viable WNV. Plaque neutralization studies involve mixing patient antibody with the virus, plating the virus/antibody mixture on Vero cells, then counting infected Vero cells (Plaque reduction test). This test is highly subjective, time consuming, and results may vary from laboratory to laboratory. Polymerase chain reaction-based (PCR) tests can be used on cerebrospinal fluid but the sensitivity and specificity of these tests is as yet poorly defined.

Peptide 0008 is a strong contender for a WNV-specific diagnostic assay, as it was only 80% conserved (eight out of ten amino acids) in Kunjin and was less well conserved in other members of the flavivirus family. Results of these studies suggest that Peptide 0008, a strong binder that scored in the range of EpiMatrix scores previously determined to be compatible with immunogenicity, would be a reasonable first candidate for the development of a tetramer-based diagnostic reagent for WNV. See, e.g., 3in X., et al., AIDS Res. Hum. Retroviruses, 16: 67-76 (2000).

WNVX Nucleic Acids and Polypeptides

The West Nile Virus nucleic acids and polypeptides of the invention, as well as derivatives, homologs, analogs and fragments thereof, will hereinafter be collectively designated as “WNVX” nucleic acid or polypeptide sequences.

One aspect of the invention pertains to isolated nucleic acid molecules that encode WNVX polypeptides or biologically active portions thereof. Also included in the invention are nucleic acid fragments sufficient for use as hybridization probes to identify WNVX-encoding nucleic acids (e.g., WNVX mRNAs and cDNAs) and fragments for use as PCR primers for the amplification and/or mutation of WN nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised of double-stranded DNA.

A WNVX nucleic acid can encode a mature WNVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of non-limiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a. proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated WNVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, or a complement of this aforementioned nucleotide sequence, can be isolated using standard molecular biology techniques and the sequence information provided herein. Using all or a portion of the nucleic acid sequence of as a hybridization probe, WNVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, NY, 1989; and Ausubel, et al., (eds.), CURRENT PROTOCOLS IN MOLECULAR BIOLOGY, John Wiley & Sons, New York, NY, 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to WNVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides or a complement thereof. Oligonucleotides may be chemically synthesized and may also be used as probes.

In another embodiment, an isolated nucleic acid molecule of the invention comprises a nucleic acid molecule that is a complement of the nucleotide sequence, or a portion of this nucleotide sequence (e.g., a fragment that can be used as a probe or primer or a fragment encoding a biologically-active portion of a WNVX polypeptide). A nucleic acid molecule that is complementary to the nucleotide sequence is one that is sufficiently complementary to the nucleotide sequence that it can hydrogen bond with little or no mismatches to the nucleotide sequence, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, NY, 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of WNVX polypeptides. Isoforms can be expressed in different tissues of the same organism as a result of, for example, alternative splicing of RNA. Alternatively, isoforms can be encoded by different genes. Homologous nucleotide sequences also include, but are not limited to, naturally occurring allelic variations and mutations of the nucleotide sequences set forth herein. A homologous nucleotide sequence does not, however, include the exact nucleotide sequence encoding WNVX protein. Homologous nucleic acid sequences include those nucleic acid sequences that encode conservative amino acid substitutions, as well as a polypeptide possessing WNVX biological activity. Various biological activities of the WNVX proteins are described below.

A WNVX polypeptide is encoded by the open reading frame (“ORF”) of a WNVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the WNVX genes allows for the generation of probes and primers designed for use in identifying and/or cloning WNVX homologues in other cell types, e.g. from other tissues. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence, or an anti-sense strand nucleotide sequence, or of a naturally occurring mutant.

Probes based on the WNVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express a WNVX protein, such as by measuring a level of a WNVX-encoding nucleic acid in a sample of cells from a subject e.g., detecting WNVX mRNA levels or determining whether a genomic WNVX gene has been mutated or deleted.

“A polypeptide having a biologically-active portion of a WNVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of WNVX” can be prepared by isolating a portion of a nucleic acid sequence that encodes a polypeptide having a WNVX biological activity (the biological activities of the WNVX proteins are described below), expressing the encoded portion of WNVX protein (e.g., by recombinant expression in vitro) and assessing the activity of the encoded portion of WNVX.

WNVX Nucleic Acid and Polypeptide Variants

The invention further encompasses nucleic acid molecules that differ from the nucleotide sequences of the invention due to degeneracy of the genetic code and thus encode the same WNVX proteins as that encoded by the nucleotide sequences of the invention. In another embodiment, an isolated nucleic acid molecule of the invention has a nucleotide sequence encoding a protein having an amino acid sequence shown in SEQ ID NOS:1-95. (See Table 1).

In addition to the WNVX nucleotide sequences, it will be appreciated by those skilled in the art that DNA sequence polymorphisms that lead to changes in the amino acid sequences of the WNVX polypeptides may exist within a population. Such genetic polymorphism in the WNVX genes may exist due to natural allelic variation. As used herein, the terms “gene” and “recombinant gene” refer to nucleic acid molecules comprising an open reading frame (ORP) encoding a WNVX protein, preferably a vertebrate WNVX protein. Such natural allelic variations can typically result in 1-5% variance in the nucleotide sequence of the WNVX genes. Any and all such nucleotide variations and resulting amino acid polymorphisms in the WNVX polypeptides, which are the result of natural allelic variation and that do not alter the functional activity of the WNVX polypeptides, are intended to be within the scope of the invention.

Nucleic acid molecules corresponding to natural allelic variants and homologues of the WNVX cDNAs of the invention can be isolated based on their homology to the WNVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid of the invention. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences of the invention corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of the invention or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of the invention or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of WNVX sequences that may exist in the population, the skilled artisan will further appreciate that changes can be introduced by mutation into the nucleotide sequences of the invention thereby leading to changes in the amino acid sequences of the encoded WNVX proteins, without altering the functional ability of said WNVX proteins. For example, nucleotide substitutions leading to amino acid substitutions at “non-essential” amino acid residues can be made. A “non-essential” amino acid residue is a residue that can be altered from the wild-type sequences of the WNVX proteins without altering their biological activity, whereas an “essential” amino acid residue is required for such biological activity. For example, amino acid residues that are conserved among the WNVX proteins of the invention are predicted to be particularly non-amenable to alteration. Amino acids for which conservative substitutions can be made are well-known within the art.

Another aspect of the invention pertains to nucleic acid molecules encoding WNVX proteins that contain changes in amino acid residues that are not essential for activity. Such WNVX proteins differ in amino acid sequence from SEQ ID NOS: 1-95 yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NOS: 1-95. Preferably, the protein encoded by the nucleic acid molecule is at least about 60% homologous to SEQ ID NOS: 1-95; more preferably at least about 70% homologous to SEQ ID NOS: 1-95; still more preferably at least about 80% homologous to SEQ ID NOS: 1-95; even more preferably at least about 90% homologous to SEQ ID NOS: 1-95; and most preferably at least about 95% homologous to SEQ ID NOS: 1-95.

An isolated nucleic acid molecule encoding a WNVX protein homologous to the protein of SEQ ID NOS: 1-95 can be created by introducing one or more nucleotide substitutions, additions or deletions into the nucleotide sequence such that one or more amino acid substitutions, additions or deletions are introduced into the encoded protein.

Mutations can be introduced into by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the WNVX protein is replaced with another amino acid residue from the same side chain family. Alternatively, in another embodiment, mutations can be introduced randomly along all or part of a WNVX coding sequence, such as by saturation mutagenesis, and the resultant mutants can be screened for WNVX biological activity to identify mutants that retain activity. Following mutagenesis, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, PYW, wherein the single letter amino acid codes are grouped by those amino acids that may be substituted for each other. Likewise, the “weak” group of conserved residues may be any one of the following: CSA, ATV, SAG, STNK, STPA, SOND, SNDEQK, NDEQHK, NEQHRK, HEY, wherein the letters within each group represent the single letter amino acid code.

In one embodiment, a mutant WNVX protein can be assayed for (i) the ability to form protein:protein interactions with other WNVX proteins, other cell-surface proteins, or biologically-active portions thereof, (ii) complex formation between a mutant WNVX protein and a WNVX ligand; or (iii) the ability of a mutant WNVX protein to bind to an intracellular target protein or biologically-active portion thereof; (e.g. avidin proteins).

In yet another embodiment, a mutant WNVX protein can be assayed for the ability to regulate a specific biological function (e.g., regulation of insulin release).

WNVX Polynucleotides Encoding WNVX Candidate Peptides

In one embodiment, the WNVX polynucleotides of the invention encode WNVX vaccine candidate peptides and express the WNVX vaccine candidate peptide in vitro in a host cell culture. The expressed WNVX vaccine candidate peptide immunogens, after suitable purification methods known to those of ordinary skill in the art, can then be incorporated into a pharmaceutical reagent or vaccine.

Alternatively, a WNVX polynucleotide encoding a WNVX vaccine candidate peptide immunogen can be administered directly into a human as so-called “naked DNA” to express the peptide immunogen in vivo in a patient. (see, Cohen, Science 259:1691 (1993); Fynan et al., Proc. Nad. Acad. Sci. USA, 90:11478 (1993); and Wolff et al., BioTechniques 11:474 (1991)). As used herein, the term “naked DNA” refers to DNA stripped of accompanying proteins or modifications. “Naked DNA” further refers to DNA not encapsulated by a liposome or virus. A WNVX polynucleotide encoding a WNVX vaccine candidate peptide immunogen can be used for direct injection into the host. This results in expression of a WNVX vaccine candidate peptide by host cells and subsequent presentation to the immune system to induce anti-WNVX antibody formation in vivo.

Determination of the sequences for the polynucleotide coding region that codes for the WNVX vaccine candidate peptides described herein can be performed using commercially available computer programs, such as DNA Strider and Wisconsin GCG or any other methods known to those skilled in the relevant arts. Due to the natural degeneracy of the genetic code, the skilled artisan will recognize that a sizable yet definite number of DNA sequences can be constructed which encode the claimed peptides (see, Watson et al., Molecular Biology of the Gene, 436-437 (the Benjamin/Cummings Publishing Co. 1987)).

Antisense Nucleic Acids

Another aspect of the invention pertains to isolated antisense nucleic acid molecules that are hybridizable to or complementary to the nucleic acid molecule comprising the nucleotide sequence, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire WNVX coding strand, or to only a portion thereof. Nucleic acid molecules encoding fragments, homologs, derivatives and analogs of a WNVX protein of SEQ ID NOS: 1-95, or antisense nucleic acids complementary to a WNVX nucleic acid are additionally provided.

In one embodiment, an antisense nucleic acid molecule is antisense to a “coding region” of the coding strand of a nucleotide sequence encoding a WNVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the WNVX protein. The term “noncoding region” refers to 5′ and 3′ sequences which flank the coding region that are not translated into amino acids (i.e., also referred to as 5′ and 3′ untranslated regions).

Given the coding strand sequences encoding the WNVX protein disclosed herein, antisense nucleic acids of the invention can be designed according to the rules of Watson and Crick or Hoogsteen base pairing. The antisense nucleic acid molecule can be complementary to the entire coding region of WNVX mRNA, but more preferably is an oligonucleotide that is antisense to only a portion of the coding or noncoding region of WNVX mRNA. For example, the antisense oligonucleotide can be complementary to the region surrounding the translation start site of WNVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a WNVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave WNVX mRNA transcripts to thereby inhibit translation of WNVX mRNA. A ribozyme having specificity for a WNVX-encoding nucleic acid can be designed based upon the nucleotide sequence of a WNVX cDNA disclosed herein. For example, a derivative of a Tetrahymena L-19 IVS RNA can be constructed in which the nucleotide sequence of the active site is complementary to the nucleotide sequence to be cleaved in a WNVX-encoding MRNA. See, e.g., U.S. Pat. No. 4,987,071 to Cech, et al. and U.S. Pat. No. 5,116,742 to Cech, et al. WNVX mRNA can also be used to select a catalytic RNA having a specific ribonuclease activity from a pool of RNA molecules. See, e.g., Bartel et al., (1993) Science 261:1411-1418.

Alternatively, WNVX gene expression can be inhibited by targeting nucleotide sequences complementary to the regulatory region of the WNVX nucleic acid (e.g., the WNVX promoter and/or enhancers) to form triple helical structures that prevent transcription of the WNVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the WNVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of WNVX can be used in therapeutic and diagnostic applications. For example, PNAs can be used as antisense or antigene agents for sequence-specific modulation of gene expression by, e.g., inducing transcription or translation arrest or inhibiting replication. PNAs of WNVX can also be used, for example, in the analysis of single base pair mutations in a gene (e.g., PNA directed PCR clamping; as artificial restriction enzymes when used in combination with other enzymes, e.g., S₁ nucleases (see, Hyrup, et al., 1996.supra); or as probes or primers for DNA sequence and hybridization (see, Hyrup, et al., 1996, supra; Perry-O'Keefe, et al., 1996. supra).

In another embodiment, PNAs of WNVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of WNVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17; 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. W088/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

WNVX Polypeptides

A polypeptide according to the invention includes a polypeptide including the amino acid sequence of WNVX polypeptides whose sequences are provided in SEQ ID NOS: 1-95. The invention also includes a mutant or variant protein any of whose residues may be changed from the corresponding residues shown in SEQ ID NOS: 1-95 while still encoding a protein that maintains its WNVX activities and physiological functions, or a functional fragment thereof.

In general, a WNVX variant that preserves WNVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated WNVX proteins, and biologically-active portions thereof, or derivatives, fragments, analogs or homologs thereof. Also provided are polypeptide fragments suitable for use as immunogens to raise anti-WNVX antibodies. In one embodiment, native WNVX proteins can be isolated from cells or tissue sources by an appropriate purification scheme using standard protein purification techniques. In another embodiment, WNVX proteins are produced by recombinant DNA techniques. Alternative to recombinant expression, a WNVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the WNVX protein is derived, or substantially free from chemical precursors or other chemicals when chemically synthesized. The language “substantially free of cellular material” includes preparations of WNVX proteins in which the protein is separated from cellular components of the cells from which it is isolated or recombinantly-produced. In one embodiment, the language “substantially free of cellular material” includes preparations of WNVX proteins having less than about 30% (by dry weight) of non-WNVX proteins (also referred to herein as a “contaminating protein”), more preferably less than about 20% of non-WNVX proteins, still more preferably less than about 10% of non-WNVX proteins, and most preferably less than about 5% of non-WNVX proteins. When the WNVX protein or biologically-active portion thereof is recombinantly-produced, it is also preferably substantially free of culture medium, ie., culture medium represents less than about 20%, more preferably less than about 10%, and most preferably less than about 5% of the volume of the WNVX protein preparation.

The language “substantially free of chemical precursors or other chemicals” includes preparations of WNVX proteins in which the protein is separated from chemical precursors or other chemicals that are involved in the synthesis of the protein. In one embodiment, the language “substantially free of chemical precursors or other chemicals” includes preparations of WNVX proteins having less than about 30% (by dry weight) of chemical precursors or non-WNVX chemicals, more preferably less than about 20% chemical precursors or non-WNVX chemicals, still more preferably less than about 10% chemical precursors or non-WNVX chemicals, and most preferably less than about 5% chemical precursors or non-WNVX chemicals.

Biologically-active portions of WNVX proteins include peptides comprising amino acid sequences sufficiently homologous to or derived from the amino acid sequences of the WNVX proteins (e.g., the amino acid sequence shown in SEQ ID NOS: 1-95) that include fewer amino acids than the full-length WNVX proteins, and exhibit at least one activity of a WNVX protein. Typically, biologically-active portions comprise a domain or motif with at least one activity of the WNVX protein. A biologically-active portion of a WNVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native WNVX protein.

In an embodiment, the WNVX protein has an amino acid sequence shown in SEQ ID NOS: 1-95. In other embodiments, the WNVX protein is substantially homologous to SEQ ID NOS: 1-95, and retains the functional activity of the protein of SEQ ID NOS: 1-95, yet differs in amino acid sequence due to natural allelic variation or mutagenesis, as described in detail, below. Accordingly, in another embodiment, the WNVX protein is a protein that comprises an amino acid sequence at least about 45% homologous to the amino acid sequence of SEQ ID NOS: 1-95, and retains the functional activity of the WNVX proteins of SEQ ID NOS: 1-95.

WNVX Peptides as Antigens

The WNVX vaccine candidate peptides can be used as antigens for raising anti-WNVX immune responses, such as T cell responses (cytotoxic T cells or T helper cells). An “antigen” is a molecule or a portion of a molecule capable of stimulating an immune response, which is additionally capable of inducing an animal or human to produce antibody capable of binding to an epitope of that antigen. An “epitope” is that portion of any molecule capable of being recognized by and bound by a major histocompatability complex (“MHC”) molecule and recognized by a T cell or bound by an antibody. A typical antigen can have one or more than one epitope. The specific recognition indicates that the antigen will react, in a highly selective manner, with its corresponding MHC and T cell, or antibody and not with the multitude of other antibodies which can be evoked by other antigens.

A peptide is “immunologically reactive” with a T cell or antibody when it binds to an MHC and is recognized by a T cell or binds to an antibody due to recognition (or the precise fit) of a specific epitope contained within the peptide. Immunological reactivity can be determined by measuring T cell response in vitro or by antibody binding, more particularly by the kinetics of antibody binding, or by competition in binding using known peptides containing an epitope against which the antibody or T cell response is directed as competitors.

Techniques used to determine whether a peptide is immunologically reactive with a T cell or with an antibody are known in the art. Peptides can be screened for efficacy by in vitro and in vivo assays. Such assays employ immunization of an animal, e.g., a rabbit or a primate, with the peptide, and evaluation of titers antibody to WNVX or to synthetic detector peptides corresponding to variant WNVX sequences. Methods of determining the spatial conformation of amino acids are known in the art, and include, for example, x-ray crystallography and 2-dimensional nuclear magnetic resonance.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides WNVX chimeric or fusion proteins. As used herein, a WNVX “chimeric protein” or “fusion protein” comprises a WNVX polypeptide operatively-linked to a non-WNVX polypeptide. An “WNVX polypeptide” refers to a polypeptide having an amino acid sequence corresponding to a WNVX protein (SEQ ID NOS: 1-95), whereas a “non-WNVX polypeptide” refers to a polypeptide having an amino acid sequence corresponding to a protein that is not substantially homologous to the WNVX protein, e.g., a protein that is different from the WNVX protein and that is derived from the same or a different organism. Within a WNVX fusion protein the WNVX polypeptide can correspond to all or a portion of a WNVX protein. In one embodiment, a WNVX fusion protein comprises at least one biologically-active portion of a WNVX protein. In another embodiment, a WNVX fusion protein comprises at least two biologically-active portions of a WNVX protein. In yet another embodiment, a WNVX fusion protein comprises at least three biologically-active portions of a WNVX protein. Within the fusion protein, the term “operatively-linked” is intended to indicate that the WNVX polypeptide and the non-WNVX polypeptide are fused in-frame with one another. The non-WNVX polypeptide can be fused to the N-terminus or C-terminus of the WNVX polypeptide.

In one embodiment, the fusion protein is a GST-WNVX fusion protein in which the WNVX sequences are fused to the C-terminus of the GST (glutathione S-transferase) sequences. Such fusion proteins can facilitate the purification of recombinant WNVX polypeptides.

In another embodiment, the fusion protein is a WNVX protein containing a heterologous signal sequence at its N-terminus. In certain host cells (e.g., mammalian host cells), expression and/or secretion of WNVX can be increased through use of a heterologous signal sequence.

In yet another embodiment, the fusion protein is a WNVX-immunoglobulin fusion protein in which the WNVX sequences are fused to sequences derived from a member of the immunoglobulin protein family. The WNVX-immunoglobulin fusion proteins of the invention can be incorporated into pharmaceutical compositions and administered to a subject to inhibit an interaction between a WNVX ligand and a WNVX protein on the surface of a cell, to thereby suppress WNVX-mediated signal transduction in vivo. The WNVX-immunoglobulin fusion proteins can be used to affect the bioavailability of a WNVX cognate ligand. Inhibition of the WNVX ligand/WNVX interaction may be useful therapeutically for both the treatment of flavivirus-associated disorders, as well as modulating (e.g. promoting or inhibiting) cell survival. Moreover, the WNVX-immunoglobulin fusion proteins of the invention can be used as immunogens to produce anti-WNVX antibodies in a subject, to purify WNVX ligands, and in screening assays to identify molecules that inhibit the interaction of WNVX with a WNVX ligand.

A WNVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A WNVX-encoding nucleic acid can be cloned into such an expression vector such that the fusion moiety is linked in-frame to the WNVX protein.

WNVX Agonists and Antagonists

The invention also pertains to variants of the WNVX proteins that function as either WNVX agonists (i.e., mimetics) or as WNVX antagonists. Variants of the WNVX protein can be generated by mutagenesis (e.g., discrete point mutation or truncation of the WNVX protein). An agonist of the WNVX protein can retain substantially the same, or a subset of, the biological activities of the naturally occurring form of the WNVX protein. An antagonist of the WNVX protein can inhibit one or more of the activities of the naturally occurring form of the WNVX protein by, for example, competitively binding to a downstream or upstream member of a cellular signaling cascade which includes the WNVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the WNVX proteins.

Variants of the WNVX proteins that function as either WNVX agonists (i.e., mimetics) or as WNVX antagonists can be identified by screening combinatorial libraries of mutants (e.g., truncation mutants) of the WNVX proteins for WNVX protein agonist or antagonist activity. In one embodiment, a variegated library of WNVX variants is generated by combinatorial mutagenesis at the nucleic acid level and is encoded by a variegated gene library. A variegated library of WNVX variants can be produced by, for example, enzymatically ligating a mixture of synthetic oligonucleotides into gene sequences such that a degenerate set of potential WNVX sequences is expressible as individual polypeptides, or alternatively, as a set of larger fusion proteins (e.g., for phage display) containing the set of WNVX sequences therein. There are a variety of methods which can be used to produce libraries of potential WNVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential WNVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the WNVX protein coding sequences can be used to generate a variegated population of WNVX fragments for screening and subsequent selection of variants of a WNVX protein. In one embodiment, a library of coding sequence fragments can be generated by treating a double stranded PCR fragment of a WNVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with Si nuclease, and ligating the resulting fragment library into an expression vector. By this method, expression libraries can be derived which encodes N-terminal and internal fragments of various sizes of the WNVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of WNVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify WNVX variants. See, e.g., Arkin and Yourvan, 1992. Proc. Natl. Acad. Sci. USA 89: 7811-7815; Delgrave, et al., 1993. Protein Engineering 6:327-331.

Anti-WNVX Antibodies

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated WNVX protein of the invention intended to serve as an antigen, or a portion or fragment thereof, can be used as an immunogen to generate antibodies that immunospecifically bind the antigen, using standard techniques for polyclonal and monoclonal antibody preparation. The full-length WNVX protein can be used or, alternatively, the invention provides antigenic peptide fragments of the antigen for use as immunogens. A WNVX antigenic peptide fragment comprises at least 6 amino acid residues of the amino acid sequence of the full length protein, such as an amino acid sequence shown in SEQ ID NOS:1-95, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of WNVXX that is located on the surface of the protein, e.g., a hydrophilic region. A hydrophobicity analysis of the human WNVXX protein sequence will indicate which regions of a WNVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a WNVX protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, NY, incorporated herein by reference). Some of these antibodies are discussed below.

1. Polyclonal Antibodies

For the production of polyclonal antibodies, various suitable host animals (e.g., rabbit, goat, mouse or other mammal) may be immunized by one or more injections with the native WNVX protein, a synthetic variant thereof, or a derivative of the foregoing. An appropriate immunogenic preparation can contain, for example, the naturally occurring immunogenic WNVX protein, a chemically synthesized polypeptide representing the immunogenic protein, or a recombinantly expressed immunogenic protein. Furthermore, the WNVX protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

2. Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding,1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

3. Humanized Antibodies

The antibodies directed against the WNVX protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

4. Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol. 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

5. F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal Fab fragments with the desired specificity for a protein or derivatives, fragments, analogs or homologs thereof. Antibody fragments that contain the idiotypes to a protein antigen may be produced by techniques known in the art including, but not limited to: (i) an F_{(ab)2} fragment produced by pepsin digestion of an antibody molecule; (ii) an F_{ab} fragment generated by reducing the disulfide bridges of an F_{(ab)2} fragment; (iii) an F_{ab} fragment generated by the treatment of the antibody molecule with papain and a reducing agent and (iv) Fᵥ fragments.

6. Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J.3 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the WNVX protein antigen described herein and further binds tissue factor (TF).

7. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl4mercaptobutyrimidate and those disclosed, for example, in U.S. Pat. No. 4,676,980.

8. Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. ExP Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design. 3: 219-230 (1989).

9. Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹i, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanedianiine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

10. Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al ., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

11. Diagnostic Applications of Antibodies Directed Against the Proteins of the Invention

Antibodies directed against a VWNVX protein of the invention may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the WNVX proteins, or derivatives, fragments, analogs or homologs thereof, that contain the antigen binding domain, are utilized as pharmacologically-active compounds (see below).

An antibody specific for a WNVX protein of the invention can be used to isolate the protein by standard techniques, such as immunoaffinity chromatography or immunoprecipitation. Such an antibody can facilitate the purification of the natural WNVX protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the WNVX protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹i, ³⁵s or ³H.

12. Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

13. Pharmaceutical Compositions of Antibodies

Antibodies specifically binding a WNVX protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic WNVX protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

14. ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte MRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, NJ, 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

WNVX Recombinant Expression Vectors and Host Cells

Another aspect of the invention pertains to vectors, preferably expression vectors, containing a nucleic acid encoding a WNVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., WNVX proteins, mutant forms of WNVX proteins, fusion proteins, etc.).

The recombinant expression vectors of the invention can be designed for expression of WNVX proteins in prokaryotic or eukaryotic cells. For example, WNVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the WNVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, WNVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banetji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to WNVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, WNVX protein can be expressed in bacterial cells such as E. coli, insect cells, yeast or mammalian cells (such as Chinese hamster ovary cells (CHO) or COS cells). These hosts can be used in connection with poxvirus vectors, such as vaccinia or swinepox. Suitable non-pathogenic viruses, which can be engineered to carry the synthetic gene into the cells of the host include poxviruses, such as vaccinia, adenovirus, retroviruses and the like. A number of such non-pathogenic viruses are commonly used for human gene therapy, and as carrier for other vaccine agents, and are known and selectable by one of skill in the art. Another preferred system includes the baculovirus expression system and vectors. Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding WNVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) WNVX protein. Accordingly, the invention further provides methods for producing WNVX protein using the host cells of the invention. In one embodiment, the method comprises culturing the host cell of invention (into which a recombinant expression vector encoding WNVX protein has been introduced) in a suitable medium such that WNVX protein is produced. In another embodiment, the method further comprises isolating WNVX protein from the medium or the host cell.

Transgenic WNVX Animals

The host cells of the invention can also be used to produce non-human transgenic animals. For example, in one embodiment, a host cell of the invention is a fertilized oocyte or an embryonic stem cell into which WNVX protein-coding sequences have been introduced. Such host cells can then be used to create non-human transgenic animals in which exogenous WNVX sequences have been introduced into their genome or homologous recombinant animals in which endogenous WNVX sequences have been altered. Such animals are useful for studying the function and/or activity of WNVX protein and for identifying and/or evaluating modulators of WNVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous WNVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing WNVX-encoding nucleic acid into the male pronuclei of a fertilized oocyte (e.g., by microinjection, retroviral infection) and allowing the oocyte to develop in a pseudopregnant female foster animal. The WNVX cDNA sequences of the invention can be introduced as a transgene into the genome of a non-human animal. Alternatively, a non-human homologue of the WNVX gene, such as a mouse WNVX gene, can be isolated based on hybridization to the WNVX cDNA (described further supra) and used as a transgene. Intronic sequences and polyadenylation signals can also be included in the transgene to increase the efficiency of expression of the transgene. A tissue-specific regulatory sequence(s) can be operably-linked to the WNVX transgene to direct expression of WNVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the WNVX transgene in its genome and/or expression of WNVX mRNA in tissues or cells of the animals. A transgenic founder animal can then be used to breed additional animals carrying the transgene. Moreover, transgenic animals carrying a transgene-encoding WNVX protein can further be bred to other transgenic animals carrying other transgenes.

To create a homologous recombinant animal, a vector is prepared which contains at least a portion of a WNYX gene into which a deletion, addition or substitution has been introduced to thereby alter, e.g., functionally disrupt, the WNVX gene. In one embodiment, the vector is designed such that, upon homologous recombination, the endogenous WNVX gene is functionally disrupted (ie., no longer encodes a functional protein; also referred to as a “knock out” vector).

Alternatively, the vector can be designed such that, upon homologous recombination, the endogenous WNVX gene is mutated or otherwise altered but still encodes functional protein (e.g., the upstream regulatory region can be altered to thereby alter the expression of the endogenous WNVX protein). In the homologous recombination vector, the altered portion of the WNVX gene is flanked at its 5′- and 3′-termini by additional nucleic acid of the WNVX gene to allow for homologous recombination to occur between the exogenous WNVX gene carried by the vector and an endogenous WNVX gene in an embryonic stem cell. The additional flanking WNVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is then introduced into an embryonic stem cell line (e.g., by electroporation) and cells in which the introduced WNVX gene has homologously-recombined with the endogenous WNVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The WNVX nucleic acid molecules, WNVX proteins, and anti-WNVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can-be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a WNVX protein or anti-WNVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. In one embodiment, the active compounds may be incorporated into lypoamino acid conjugates as described in Toth, et al., J. Drug Target, 2:217-239 (1994). Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The vaccine composition can include as the active agents, one of the following components: (a) an WNVX vaccine candidate peptide immunogen, which can be in the form of recombinant proteins or, alternatively, can be in the form of a mixture of carrier protein conjugates; (b) a polynucleotide encoding a WNVX vaccine candidate; (c) a recombinant virus carrying the synthetic gene or molecule; and (d) a bacteria carrying the WNVX vaccine candidate. The selected active component may be present in a pharmaceutically acceptable carrier, and the vaccine composition can also contain additional ingredients.

Formulations containing the WNVX vaccine candidate peptide may also contain other active agents, including, but not limited to, such as adjuvants and immunostimulatory cytokines, such as IL-12 and other well-known cytokines, for the vaccine compositions.

WNVX vaccine candidate peptide immunogens may be linked to a suitable carrier in order to improve the efficacy of antigen presentation to the immune system. Such carriers can be, for instance, organic polymers. A carrier protein can enhance the immunogenicity of the peptide immunogen. Such a carrier can be a larger molecule, which has an adjuvant effect. Exemplary conventional protein carriers include, keyhole limpet hemocyan, E. coli DnaK protein, galactokinase (galK, which catalyzes the first step of galactose metabolism in bacteria), ubiquitin, α-mating factor, β-galactosidase, and influenza NS-1 protein. Toxoids (i.e., nucleic acid sequences which encode the naturally occurring toxin, with sufficient modifications to eliminate its toxic activity) such as diphtheria toxoid and tetanus toxoid can also be employed as carriers. Similarly, a variety of bacterial heat shock proteins, e.g., mycobacterial hsp-70, can be used. Glutathione reductase (GST) is another useful carrier. One of skill in the art can readily determine and select an appropriate carrier.

Viruses can be modified by recombinant DNA technology such as, e.g. rhinovirus, poliovirus, vaccinia, or influenzavirus, etc. The peptide can be linked to a modified, i.e., attenuated or recombinant virus such as modified influenza virus or modified hepatitis B virus or to parts of a virus, e.g., to a viral glycoprotein such as, e.g., hemagglutinin of influenza virus or surface antigen of hepatitis B virus, in order to increase the immunological response against WNVX-infected cells.

Other antigen carrier systems may also be used to enhance immunogenicity. In one embodiment, the immunogens of the invention are incorporated into a multi-peptide conjugate (MPC) system, where the immunogens are synthesized and coupled to a core peptide template using known methods of peptide synthesis and solution chemistry. See e.g., Boykins, et al., Peptides 21:9-17 (2000). In another embodiment, the immunogens are incorporated into a Multiple Antigen Peptide System (MAPS) as described by Tam in U.S. Pat. No. 5,229,490.

Where the polynucleotides of the invention are naked DNA vaccines, suitable vehicles for direct DNA, plasmid polynucleotide, or recombinant vector administration include, without limitation, saline, sucrose, protamine, polybrene, polylysine, polycations, proteins, calcium phosphate, or spermidine. See e.g, PCT International patent application WO 94/01139. As with the immunogenic compositions, the amounts of components in the DNA and vector compositions and the mode of administration, e.g., injection or intranasal, can be selected and modified by one of skill in the art. Generally, each dose will comprise between about 50 μg to about 1 mg of immunogen-encoding DNA per ml of a sterile solution.

For recombinant viruses containing the coding polynucleotide, the doses can range from about 20 to about 50 ml of saline solution containing concentrations of from about 1×10⁷ to 1×10¹⁰ pfu/ml recombinant virus of the invention. One suitable human dosage is about 20 ml saline solution at the above concentrations. However, it is understood that one of skill in the art can alter such dosages depending upon the identity of the recombinant virus and the make-up of the immunogen that it is being delivered to the host.

The amounts of the commensal bacteria carrying the synthetic gene or molecules to be delivered to the patient will generally range between about 10³ to about 10¹² cells/kg. These dosages, will of course, be altered by one of skill in the art depending upon the bacterium being used and the particular composition containing immunogens being delivered by the live bacterium.

The pharmaceutical and vaccine compositions of the invention can be included in a container, pack, or dispenser together with instructions for administration.

Screening and Detection Methods

The isolated nucleic acid molecules of the invention can be used to express WNVX protein (e.g., via a recombinant expression vector in a host cell in gene therapy applications), to detect WNVX mRNA (e.g., in a biological sample) or a genetic lesion in a WNVX gene, and to modulate WNVX activity, as described further, below. In addition, the WNVX proteins can be used to screen drugs or compounds that modulate the WNVX protein activity or expression. In addition, the anti-WNVX antibodies of the invention can be used to detect and isolate WNVX proteins and modulate WNVX activity.

The invention further pertains to novel agents identified by the screening assays described herein and uses thereof for treatments as described, supra.

Screening Assays

The invention provides a method (also referred to herein as a “screening assay”) for identifying modulators, ie., candidate or test compounds or agents (e.g., peptides, peptidomimetics, small molecules or other drugs) that bind to WNVX proteins or have a stimulatory or inhibitory effect on, e.g., WNVX protein expression or WNVX protein activity. The invention also includes compounds identified in the screening assays described herein.

In one embodiment, the invention provides assays for screening candidate or test compounds which bind to or modulate the activity of the membrane-bound form of a WNVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406;.Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of WNVX protein, or a biologically-active portion thereof, on the cell surface is contacted with a test compound and the ability of the test compound to bind to a WNVX protein determined. The cell, for example, can of mammalian origin or a yeast cell. Determining the ability of the test compound to bind to the WNVX protein can be accomplished, for example, by coupling the test compound with a radioisotope or enzymatic label such that binding of the test compound to the WNVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of WNVX protein, or a biologically-active portion thereof, on the cell surface with a known compound which binds WNVX to form an assay mixture, contacting the assay mixture with a test compound, and determining the ability of the test compound to interact with a WNVX protein, wherein determining the ability of the test compound to interact with a WNVX protein comprises determining the ability of the test compound to preferentially bind to WNVX protein or a biologically-active portion thereof as compared to the known compound.

In another embodiment, an assay is a cell-based assay comprising contacting a cell expressing a membrane-bound form of WNVX protein, or a biologically-active portion thereof, on the cell surface with a test compound and determining the ability of the test compound to modulate (e.g., stimulate or inhibit) the activity of the WNVX protein or biologically-active portion thereof. Determining the ability of the test compound to modulate the activity of WNVX or a biologically-active portion thereof can be accomplished, for example, by determining the ability of the WNVX protein to bind to or interact with a WNVX target molecule. As used herein, a “target molecule” is a molecule with which a WNVX protein binds or interacts in nature, for example, a molecule on the surface of a cell which expresses a WNVX interacting protein, a molecule on the surface of a second cell, a molecule in the extracellular milieu, a molecule associated with the internal surface of a cell membrane or a cytoplasmic molecule. A WNVX target molecule can be a non-WNVX molecule or a WNVX protein or polypeptide of the invention. In one embodiment, a WNVX target molecule is a component of a signal transduction pathway that facilitates transduction of an extraceliular signal (e.g. a signal generated by binding of a compound to a membrane-bound WNVX molecule) through the cell membrane and into the cell. The target, for example, can be a second intercellular protein that has catalytic activity or a protein that facilitates the association of downstream signaling molecules with WNVX.

Determining the ability of the WNVX protein to bind to or interact with a WNVX target molecule can be accomplished by one of the methods described above for determining direct binding. In one embodiment, determining the ability of the WNVX protein to bind to or interact with a WNVX target molecule can be accomplished by determining the activity of the target molecule. For example, the activity of the target molecule can be determined by detecting induction of a cellular second messenger of the target (ie. intracellular Ca²⁺, diacylglycerol, IP₃, etc.), detecting catalytic/enzymatic activity of the target an appropriate substrate, detecting the induction of a reporter gene (comprising a WNVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a WNVX protein or biologically-active portion thereof with a test compound and determining the ability of the test compound to bind to the WNVX protein or biologically-active portion thereof Binding of the test compound to the WNVX protein can be determined either directly or indirectly as described above. In one such embodiment, the assay comprises contacting the WNVX protein or biologically-active portion thereof with a known compound which binds WNVX to form an assay mixture, contacting the assay mixture with a test compound, and determining the ability of the test compound to interact with a WNVX protein, wherein determining the ability of the test compound to interact with a WNVX protein comprises determining the ability of the test compound to preferentially bind to WNVX or biologically-active portion thereof as compared to the known compound.

In still another embodiment, an assay is a cell-free assay comprising contacting WNVX protein or biologically-active portion thereof with a test compound and determining the ability of the test compound to modulate (e.g. stimulate or inhibit) the activity of the WNVX protein or biologically-active portion thereof. Determining the ability of the test compound to modulate the activity of WNVX can be accomplished, for example, by determining the ability of the WNVX protein to bind to a WNVX target molecule by one of the methods described above for determining direct binding. In an alternative embodiment, determining the ability of the test compound to modulate the activity of WNVX protein can be accomplished by determining the ability of the WNVX protein further modulate a WNVX target molecule. For example, the catalytic/enzymatic activity of the target molecule on an appropriate substrate can be determined as described, supra.

In yet another embodiment, the cell-free assay comprises contacting the WNVX protein or biologically-active portion thereof with a known compound which binds WNVX protein to form an assay mixture, contacting the assay mixture with a test compound, and determining the ability of the test compound to interact with a WNVX protein, wherein determining the ability of the test compound to interact with a WNVX protein comprises determining the ability of the WNVX protein to preferentially bind to or modulate the activity of a WNVX target molecule.

The cell-free assays of the invention are amenable to use of both the soluble form or the membrane-bound form of WNVX protein. In the case of cell-free assays comprising the membrane-bound form of WNVX protein, it may be desirable to utilize a solubilizing agent such that the membrane-bound form of WNVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either WNVX protein or its target molecule to facilitate separation of complexed from uncomplexed forms of one or both of the proteins, as well as to accommodate automation of the assay. Binding of a test compound to WNVX protein, or interaction of WNVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-WNVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or WNVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of WNVX protein binding or activity determined using standard techniques.

Other techniques for immobilizing proteins on matrices can also be used in the screening assays of the invention. For example, either the WNVX protein or its target molecule can be immobilized utilizing conjugation of biotin and streptavidin. Biotinylated WNVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with WNVX protein or target molecules, but which do not interfere with binding of the WNVX protein to its target molecule, can be derivatized to the wells of the plate, and unbound target or WNVX protein trapped in the wells by antibody conjugation. Methods for detecting such complexes, in addition to those described above for the GST-immobilized complexes, include immunodetection of complexes using antibodies reactive with the WNVX protein or target molecule, as well as enzyme-linked assays that rely on detecting an enzymatic activity associated with the WNVX protein or target molecule.

In another embodiment, modulators of WNVX protein expression are identified in a method wherein a cell is contacted with a candidate compound and the expression of WNVX mRNA or protein in the cell is determined. The level of expression of WNVX mRNA or protein in the presence of the candidate compound is compared to the level of expression of WNVX mRNA or protein in the absence of the candidate compound. The candidate compound can then be identified as a modulator of WNVX MRNA or protein expression based upon this comparison. For example, when expression of WNVX mRNA or protein is greater (Le., statistically significantly greater) in the presence of the candidate compound than in its absence, the candidate compound is identified as a stimulator of WNVX MRNA or protein expression. Alternatively, when expression of WNVX mRNA or protein is less (statistically significantly less) in the presence of the candidate compound than in its absence, the candidate compound is identified as an inhibitor of WNVX mRNA or protein expression. The level of WNVX mRNA or protein expression in the cells can be determined by methods described herein for detecting WNVX MRNA or protein.

In yet another aspect of the invention, the WNVX proteins can be used as “bait proteins” in a two-hybrid assay or three hybrid assay (see, e.g., U.S. Pat. No.5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with WNVX (“WNVX-binding proteins” or “WNVX-bp”) and modulate WNVX activity. Such WNVX-binding proteins are also likely to be involved in the propagation of signals by the WNVX proteins as, for example, upstream or downstream elements of the WNVX pathway.

The two-hybrid system is based on the modular nature of most transcription factors, which consist of separable DNA-binding and activation domains. Briefly, the assay utilizes two different DNA constructs. In one construct, the gene that codes for WNVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a WNVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with WNVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) locate gene regions associated with viral susceptibility; (ii) identify an individual from a minute biological sample (tissue typing); and (iii) aid in forensic identification of a biological sample. Some of these applications are described in the subsections, below.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining WNVX protein and/or nucleic acid expression as well as WNVX activity, in the context of a biological sample (e.g., blood, serum, cells, tissue) to thereby determine whether an individual is afflicted with a disease or disorder, or is at risk of developing a disorder, associated with WNVX infection. The disorders include flavivirus disorders such as St. Louis Encephalitis, Japanese Encephalitis, Hepatitis C, and Dengue. The invention also provides for prognostic (or predictive) assays for determining whether an individual is at risk of developing a disorder associated with WNVX protein, nucleic acid expression or activity. For example, mutations in a WNVX gene can be assayed in a biological sample. Such assays can be used for prognostic or predictive purpose to thereby prophylactically treat an individual prior to the onset of a disorder characterized by or associated with WNVX protein, nucleic acid expression, or biological activity.

Another aspect of the invention provides methods for determining WNVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the activity of WNVX in clinical trials.

These and other agents are described in further detail in the following sections.

Diagnostic Assays

An exemplary method for detecting the presence or absence of WNVX in a biological sample involves obtaining a biological sample from a test subject and contacting the biological sample with a compound or an agent capable of detecting WNVX protein or nucleic acid (e.g., MRNA, genomic DNA) that encodes WNVX protein such that the presence of WNVX is detected in the biological sample. An agent for detecting WNVX MRNA or genomic DNA is a labeled nucleic acid probe capable of hybridizing to WNVX mRNA or genomic DNA. The nucleic acid probe can be, for example, a fill-length WNVX nucleic acid, or a portion thereof, such as an oligonucleotide of at least 15, 30, 50, 100, 250 or 500 nucleotides in length and sufficient to specifically hybridize under stringent conditions to WNVX mRNA or genomic DNA. Other suitable probes for use in the diagnostic assays of the invention are described herein.

An agent for detecting WNVX protein is an antibody capable of binding to WNVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (ie., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect WNVX “mRNA, protein, or genomic DNA in a biological sample in vitro as well as in vivo. For example, in vitro techniques for detection of WNVX mRNA include Northern hybridizations and in situ hybridizations. In vitro techniques for detection of WNVX protein include enzyme linked immunosorbent assays (EUSAs), Western blots, immunoprecipitations, and immunofluorescence. In vitro techniques for detection of WNVX genomic DNA include Southern hybridizations. Furthermore, in vivo techniques for detection of WNVX protein include introducing into a subject a labeled anti-WNVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting WNVX protein, mRNA, or genomic DNA, such that the presence of WNVX protein, mRNA or genomic DNA is detected in the biological sample, and comparing the presence of WNVX protein, mRNA or genomic DNA in the control sample with the presence of WNVX protein, mRNA or genomic DNA in the test sample.

The invention also encompasses kits for detecting the presence of WNVX in a biological sample. For example, the kit can comprise: a labeled compound or agent capable of detecting WNVX protein or MRNA in a biological sample; means for determining the amount of WNVX in the sample; and means for comparing the amount of WNVX in the sample with a standard. The compound or agent can be packaged in a suitable container. The kit can further comprise instructions for using the kit to detect WNVX protein or nucleic acid.

Prognostic Assays

The diagnostic methods described herein can furthermore be utilized to identify subjects susceptible to WNVX infection. For example, the assays described herein, such as the preceding diagnostic assays or the following assays, can be utilized to identify a subject having or at risk of developing a disorder associated with WNVX protein, nucleic acid expression or activity. Alternatively, the prognostic assays can be utilized to identify a subject having or at risk for developing a disease or disorder. Thus, the invention provides a method for identifying a disease or disorder associated with WNVX infection in which a test sample is obtained from a subject and WNVX protein or nucleic acid (e.g., MRNA, genomic DNA) is detected, wherein the presence of WNVX protein or nucleic acid is diagnostic for a subject having or at risk of developing a disease or disorder associated with WNVX. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with WNVX infection. For example, such methods can be used to determine whether a subject can be effectively treated with an agent for a disorder. Thus, the invention provides methods for determining whether a subject can be effectively treated with an agent for a disorder associated with WNVX infection in which a test sample is obtained and WNVX protein or nucleic acid is detected (e.g., wherein the presence of WNVX protein or nucleic acid is diagnostic for a subject that can be administered the agent to treat a disorder associated with WNVX infection).

The methods of the invention can also be used to detect genetic lesions in a WNVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a WNVX-protein, or the misexpression of the WNVX gene. For example, such genetic lesions can be detected by ascertaining the existence of at least one of: (i) a deletion of one or more nucleotides from a WNVX gene; (ii) an addition of one or more nucleotides to a WNVX gene; (iii) a substitution of one or more nucleotides of a WNVX gene, (iv) a chromosomal rearrangement of a WNVX gene; (v) an alteration in the level of a messenger RNA transcript of a WNVX gene, (vi) aberrant modification of a WNVX gene, such as of the methylation pattern of the genomic DNA, (vii) the presence of a non-wild-type splicing pattern of a messenger RNA transcript of a WNVX gene, (viii) a non-wild-type level of a WNVX protein, (ix) allelic loss of a WNVX gene, and (x) inappropriate post-translational modification of a WNVX protein. As described herein, there are a large number of assay techniques known in the art which can be used for detecting lesions in a WNVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the WNVX-gene (see, Abravaya, et al., 1995. Nucl. Acids Res. 23: 675-682). This method can include the steps of collecting a sample of cells from a patient, isolating nucleic acid (e.g., genomic, MRNA or both) from the cells of the sample, contacting the nucleic acid sample with one or more primers that specifically hybridize to a WNVX gene under conditions such that hybridization and amplification of the WNVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a WNVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in WNVX nucleic acids can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in WNVX nucleic acids can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the WNVX gene and detect mutations by comparing the sequence of the sample WNVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Diotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the WNVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type WNVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in WNVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a WNVX sequence, e.g., a wild-type WNVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in WNVX genes. For example, single strand conformation polymorphism (SSCP) may be used to detect differences in electrophoretic mobility between mutant and wild type nucleic acids. See, e.g., Orita, et al., 1989. Proc. Nat]. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control WNVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saild, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a WNVX gene.

Furthermore, any cell type or tissue, preferably peripheral blood leukocytes, in which WNVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on WNVX activity (e.g., WNVX gene expression), as identified by a screening assay described herein can be administered to individuals to treat (prophylactically or therapeutically) disorders (The disorders include flavivirus associated disorders. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of WNVX protein, expression of WNVX nucleic acid, or mutation content of WNVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of VWNVX protein, expression of WNVX nucleic acid, or mutation content of WNVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a WNVX modulator, such as a modulator identified by one of the exemplary screening assays described herein.

Monitoring of Effects During Clinical Trials

Monitoring the influence of agents (e.g., drugs, compounds) on the expression or activity of WNVX (e.g., the ability to modulate aberrant cell proliferation and/or differentiation) can be applied not only in basic drug screening, but also in clinical trials. For example, the effectiveness of an agent determined by a screening assay as described herein to increase WNVX gene expression, protein levels, or upregulate WNVX activity, can be monitored in clinical trails of subjects exhibiting decreased WNVX gene expression, protein levels, or downregulated WNVX activity. Alternatively, the effectiveness of an agent determined by a screening assay to decrease WNVX gene expression, protein levels, or downregulate WNVX activity, can be monitored in clinical trails of subjects exhibiting increased WNVX gene expression, protein levels, or upregulated WNVX activity. In such clinical trials, the expression or activity of WNVX and, preferably, other genes that have been implicated in, for example, a cellular proliferation or immune disorder can be used as a “read out” or markers of the immune responsiveness of a particular cell.

By way of example, and not of limitation, genes, including WNVX, that are modulated in cells by treatment with an agent (e.g., compound, drug or small molecule) that modulates WNVX activity (e.g., identified in a screening assay as described herein) can be identified. Thus, to study the effect of agents on cellular proliferation disorders, for example, in a clinical trial, cells can be isolated and RNA prepared and analyzed for the levels of expression of WNVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of WNVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a WNVX protein, mRNA, or genomic DNA in the preadministration sample; (iii) obtaining one or more post-administration samples from the subject; (iv) detecting the level of expression or activity of the WNVX protein, mRNA, or genomic DNA in the post-administration samples; (v) comparing the level of expression or activity of the WNVX protein, MRNA, or genomic DNA in the pre-administration sample with the WNVX protein, mRNA, or genomic DNA in the post administration sample or samples; and (vi) altering the administration of the agent to the subject accordingly. For example, increased administration of the agent may be desirable to increase the expression or activity of WNVX to higher levels than detected, i.e., to increase the effectiveness of the agent. Alternatively, decreased administration of the agent may be desirable to decrease expression or activity of WNVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant WNVX expression or activity. The disorders include flavivirus-related disorders.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endoggenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (Le., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant WNVX expression or activity, by administering to the subject an agent that modulates WNVX expression or at least one WNVX activity. Subjects at risk for a disease that is caused or contributed to by aberrant WNVX expression or activity can be identified by, for example, any or a combination of diagnostic or prognostic assays as described herein. Administration of a prophylactic agent can occur prior to the manifestation of symptoms characteristic of the WNVX aberrancy, such that a disease or disorder is prevented or, alternatively, delayed in its progression. Depending upon the type of WNVX aberrancy, for example, a WNVX agonist or WNVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating WNVX expression or activity for therapeutic purposes. The modulatory method of the invention involves contacting a cell with an agent that modulates one or more of the activities of WNVX protein activity associated with the cell. An agent that modulates WNVX protein activity can be an agent as described herein, such as a nucleic acid or a protein, a naturally-occurring cognate ligand of a WNVX protein, a peptide, a WNVX peptidomimetic, or other small molecule. In one embodiment, the method involves administering an agent (e.g., an agent identified by a screening assay described herein), or combination of agents that modulates (e.g., up-regulates or down-regulates) WNVX expression or activity. In another embodiment, the method involves administering a WNVX protein or nucleic acid molecule as therapy to compensate for reduced or aberrant WNVX expression or activity.

In another embodiment, the agent inhibits one or more WNVX protein activity. Examples of such inhibitory agents include antisense WNVX nucleic acid molecules and anti-WNVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a WNVX protein or nucleic acid molecule. The method for reducing the levels of WNVX involves exposing a human to a WNVX vaccine candidate peptides, actively inducing antibodies that react with WNVX, and impairing the multiplication of WNVX in vivo. This method is appropriate for an WNVX infected subject with a competent immune system, or an uninfected or recently infected subject. The method induces antibodies, which react with WNVX, which reduces multiplication during any initial acute infection with WNVX.

The terms “treating,” “treatment,” and the like are used herein to mean obtaining a desired pharmacologic or physiologic effect. The effect can be prophylactic in terms of completely or partially preventing a disorder or sign or symptom thereof, or can be therapeutic in terms of a partial or complete cure for a disorder and/or adverse effect attributable to the disorder. “Treating” as used herein covers any treatment and includes: (a) preventing a disorder from occurring in a subject that can be predisposed to a disorder, but has not yet been diagnosed as having it; (b) inhibiting the disorder, i.e., arresting its development; or (c) relieving or ameliorating the disorder. An “effective amount” or “therapeutically effective amount” is the amount sufficient to obtain the desired physiological effect. An effective amount of the WNVX vaccine candidate peptide or vector expressing WNVX vaccine candidate peptides is generally determined by the physician in each case on the basis of factors normally considered by one skilled in the art to determine appropriate dosages, including the age, sex, and weight of the subject to be treated, the condition being treated, and the severity of the medical condition being treated. Among such patients suitable for treatment with this method are WNVX infected patients.

Method of Administration

WNVX vaccine candidate peptides can be administered in a variety of ways, orally, topically, parenterally e.g. subcutaneously, intraperitoneally, by viral infection, intravascularly, etc. Depending upon the manner of introduction, the WNVX vaccine candidate peptides can be formulated in a variety of ways. The concentration of WNVX vaccine candidate peptides in the formulation can vary from about 0.1-100 wt. %.

The amount of the WNVX vaccine candidate peptide or polynucleotides of the invention present in each vaccine dose is selected with regard to consideration of the patient's age, weight, sex, general physical condition and the like. The amount of WNVX vaccine candidate peptide required to induce an immune response, preferably a protective response, or produce an exogenous effect in the patient without significant adverse side effects varies depending upon the pharmaceutical composition employed and the optional presence of an adjuvant. Generally, for the compositions containing WNVX vaccine candidate peptide, each dose will comprise between about 50 Ag to about 1 mg of the WNVX vaccine candidate peptide immunogens/ml of a sterile solution. A more preferred dosage can be about 200 μg of WNVX vaccine candidate peptide immunogen. Other dosage ranges can also be contemplated by one of skill in the art Initial doses can be optionally followed by repeated boosts, where desirable. The method can involve chronically administering the WNVX vaccine candidate peptide composition. For therapeutic use or prophylactic use, repeated dosages of the immunizing compositions can be desirable, such as a yearly booster or a booster at other intervals. The dosage administered will, of course, vary depending upon known factors such as the pharmacodynamic characteristics of the particular agent, and its mode and route of administration; age, health, and weight of the recipient; nature and extent of symptoms, kind of concurrent treatment, frequency of treatment, and the effect desired. Usually a daily dosage of active ingredient can be about 0.01 to 100 mg/kg of body weight. Ordinarily 1.0 to 5, and preferably 1 to 10 mg/kg/day given in divided doses 1 to 6 times a day or in sustained release form is effective to obtain desired results.

The WNVX vaccine candidate peptide can be employed in chronic treatments for subjects at risk of acute infection. A dosage frequency for such “acute” infections may range from daily dosages to once or twice a week intravenously or intramuscularly, for a duration of about 6 weeks. The peptides can also be employed in chronic treatments for infected patients. In infected patients, the frequency of chronic administration can range from daily dosages to once or twice a week i.v. or i.m., and may depend upon the half-life of the immunogen (e.g., about 7-21 days). However, the duration of chronic treatment for such infected patients is anticipated to be an indefinite, but prolonged period.

For such therapeutic uses, the WNVX vaccine candidate peptide formulations and modes of administration are substantially identical to those described specifically above and can be administered concurrently or simultaneously with other conventional therapeutics.

In another embodiment, mulitple WNVX nucleic acids and proteins are used simultaneously as a combination vaccine. See e.g., Rennels, et al., Pediatrics, 96:576-79 (1995); Eskola, et al., Vaccine, 8(2):107-10 (1990). In an alternative embodiment, mulitple WNVX nucleic acids and proteins are utilized in multi-stage vaccine regimes, such as the prime-boost method described in Ramshaw, et al., Immunology Today, 21:4:163-65 (2000).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The WNVX nucleic acids and proteins of the invention are useful in potential prophylactic and therapeutic applications implicated in a variety of flavivirus disorders.

As an example, a cDNA encoding the WNVX protein of the invention may be useful in gene therapy, and the protein may be useful when administered to a subject in need thereof. By way of non-limiting example, the compositions of the invention will have efficacy for treatment of patients suffering from a flaviviral disorder.

Both the novel nucleic acid encoding the WNVX protein, and the WNVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies which immunospecifically-bind to the novel substances of the invention for use in therapeutic or diagnostic methods.

The invention will be further described in the following examples, which do not limit the scope of the invention described in the claims.

### EXAMPLES

### Example 1

### Obtaining the WNV Sequence

The NY 1999 WNV sequence was obtained from the Genbank (Genbank accession number AF196835). The 3,433 amino acids contained in the Genbank translation were parsed into 3,424 10-amino acid long frames, each 10 amino acid-long peptide sequence overlapping the previous peptide sequence by one amino acid (see Table 1 for an illustration). The sequences of these 3,424 10 mers were stored in a database (WNV peptide database).

Table 2 provides an example of the analysis performed to select candidate B*07 ligands from the WNV genome. The sequence of WNV was parsed into overlapping peptides, 10 amino acids in length, overlapping by one amino acid, starting with the first amino acid to be translated from the WNV sequence (AA 0001). The resulting 3,423 10 mers were then compared to the EpiMatrix B*07 matrix and evaluated for match to the matrix pattern. Peptides that best matched the matrix received the highest EBP which is the value that EpiMatrix uses to describe the probability that the peptide will bind to B*07 in vitro and in vivo. Of the 6 overlapping peptides in this particular region of the WNV sequence shown here, WNVB7 0019 received the best EMX score (22.49), and could be considered therefore the most likely candidate for in vitro studies (of this set of 6 peptides).

The 25 peptides tested in vitro are shown in Table 3a. Twenty of the peptides were selected because they had an EpiMatrix (EMX) score (estimated binding probability, or EBP) between 7 and 50. Peptides that were not tested even though they may fit the above criteria are listed in Table 3b. Three peptides received EMX scores above 50 (0001, 0002, 0003). These peptides were considered to be unlikely to be epitopes based on previous experience with HLA B*07 restricted HIV-1 epitopes (TB/HIV Research Lab, unpublished data). Additionally, two peptides were not selected, even though they were predicted to be binders (0016, 0022), because they fell in regions of the genome that were considered unlikely to be expressed, based on information provided by Genbank.

### Example 2

### EpiMatrix Analysis

EpiMatrix is a matrix-based algorithm that ranks 10 amino acid long segments, overlapping by 9 amino acids, from any protein sequence by estimated probability of binding to a selected MHC molecule. (De Groot et al., AIDS Research and Human Retroviruses 13:539-41 (1997)). The procedure for developing matrix motifs was published by Schafer et al, 16 Vaccine 1998 (1998). We have constructed matrix motifs for 32 HLA class I alleles, one murine allele (H-2 Kd) and several human class II alleles. Putative MHC ligands are selected by scoring each 10-mer frame in a protein sequence. This score, or estimated binding probability (EBP), is derived by comparing the sequence of the 10-mer to the matrix of 10 amino acid sequences known to bind to each MHC allele. Retrospective studies have demonstrated that EpiMatrix accurately predicts published MHC ligands (Jesdale et al., in Vaccines '97 (Cold Spring Harbor Press, Cold Spring Harbor, N.Y., 1997)).

An additional feature of EpiMatrix is that it can measure the MHC binding potential of each 10 amino acid long snapshot to a number of human HLA, and therefore can be used to identify regions of MHC binding potential clustering. Other laboratories have confirmed cross-presentation of peptides within HLA “superfamilies” (A11, A3, A31, A33 and A68) (Jesdale et al., in Vaccines '97 (Cold Spring Harbor Press, Cold Spring Harbor, N.Y., 1997)). Presumably, vaccines containing such “clustered” or promiscuous epitopes will have an advantage over vaccines composed of epitopes that are not “clustered. In work performed in the TB/HIV Research Lab, we have confirmed cross-MHC binding that was predicted by EpiMatrix.

Each of the peptides was then evaluated using EpiMatrix, a matrix-based algorithm that ranks 10 amino acid peptides by estimated probability of binding to a selected MHC molecule as follows. The peptides are scored by estimating the relative promotion or inhibition of binding for each amino acid, compared to known MHC binders for that allele. This information is summed across the peptide and a summary score (EMX score) is assigned to the entire peptide. After comparing the EMX score to the scores of known MHC ligands, EpiMatrix arrives at an “estimated binding probability” (abbreviated as EBP, but not strictly a probability). The EBP describes the proportion of peptides with EpiMatrix scores as high or higher that will bind to a given MHC molecule. EBPs range from 100% (highly likely to bind) to less than 1% (very unlikely to bind).

The EpiMatrix approach was used to screen the West Nile Virus NY99 genome for putative HLA B*07 restricted epitopes. Ninety-four of 3,433 WNV peptides scored above a predetermined cutoff suggesting that they would be likely to bind to HLA B*07. Sixteen of the 94 candidate B*07 ligands and four peptides that were not expected to be ligands based on their EpiMatrix score were synthesized. Twelve of the sixteen putative HLA B*07 ligands (75%) were shown stabilize HLA B*07 molecules on the surface of T2B7 cells. Five of these peptides, IPAGFEPEML (WNV B*07 0008) (SEQ ID NO: 8); RPRWIDARVY (WNV B*07 0017) (SEQ ID NO: 17); RPQRHDEKTL (WNV Be 07 0019) (SEQ ID NO: 19); SPHRVPNYNL (WNV B*07 0020) (SEQ ID NO:20); and RPAADGRTVM (WNV B*07 0023) (SEQ D NO 23) bound with much greater affinity to B*07 in vitro than another, previously published B*07 epitope GPGHKARVLA (from HIV-1) (SEQ ID NO: 96). None of four selected “non-binders” stabilized HLA B*07 to a significant degree. MHC ligands identified using this method may be used to screen T cells derived from WNV-exposed individuals for cell-mediated response to WNV or to develop diagnostic reagents such as tetramers for epidemiological surveillance.

Using the EpiMatrix approach, five excellent B*07-restricted T cell epitope candidates for an emerging infectious disease were rapidly identified. Overall, twelve of 16 (75%) peptides selected for this study bound in T2B7 binding assays.

Sixteen WNV peptides were screened. Twelve epitope candidates were identified over the course of 20 working days. Five of these candidates exhibited excellent binding to HLA B*07 in vitro, suggesting that they might be excellent reagents for developing tetramer assays. The largest source of delay in the process was peptide synthesis (four weeks from placement of order to receipt of the first set of peptides. This entire process could be accelerated, if more rapid access to MHC ligands were necessary.

The binding studies described here are a first step along the path to confirming immunogenicity, however, in cases (such as WNV) where access to T cells from infected individuals is limited, both the bioinformatics step and the binding assays can be executed without clinical specimens. Once the epitope candidates selected using this method are confirmed in CTL assays, they may be useful for (1) screening exposed individuals, (2) investigating the immuno-pathogenesis of WNV disease in humans, (3) as components of diagnostic kits developed for the surveillance effort, and/or (4) eventually, as a tool for measuring WNV vaccine-related immune responses. Confirmation of T cell response to the peptides will depend on availability of peripheral blood cells from West Nile-infected patients during the next transmission season. Additional peptides would also need to be defined and screened for binding to other HLA alleles, in order to broaden the MHC specificity of the diagnostic reagent or immunopathogenesis tools developed using this approach.

### Example 3

### Analysis of Overall Scores, Compared to a Random Set and a Set of Known HLA B*07 Binders

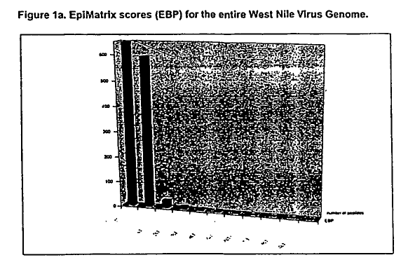

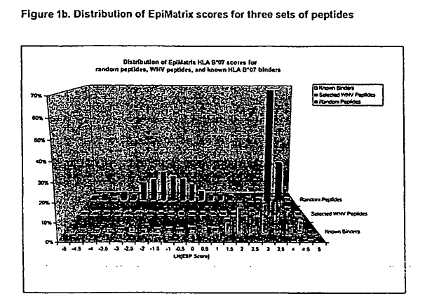

The 3,424 10 mers derived from WNV were compared to the EpiMatrix B*07 matrix and evaluated for match to the matrix pattern. The majority of decamers scored for the entire WNV genome (using the HLA B*07 scoring matrix) fell below 1% EBP score (FIG. 1a). This is also generally true for other proteins we have analyzed (Xia Jin et al., 1999, K. Bond, J. McNicholl, manuscript in preparation, and unpublished data from the TB/HIV Research Lab). FIG. 1b shows the distribution of HLA B*07 scores of a set of 10,000 random peptides (plotted as their natural logs, so as to better distribute EBP scores falling below 1), compared to scores for a set of more than 300 known binders (compiled and maintained at EpiVax) and to the scores of the set of WNV peptides selected for this study. As can be seen in the figure, the set of peptides selected for this study fell well within the EBP range for a set of more than 300 known HLA B*07 ligands.

Table 4 provides an illustration of the analysis performed to select candidate B*07 ligands from the WNV genome. Of the 6 overlapping peptides in this particular region of the WNV sequence shown here, WNVB7 0019 received the best EMX score (22.49), and could be considered therefore the most likely candidate for in vitro studies (of this set of 6 peptides).

TABLE 4

Scoring overlapping peptides using
EpiMatrix motif HLA B*07

| | | | | |
|---|---|---|---|---|
| SEQ ID | AA | peptide number | | |
| NO: | Start | (B*07 rank) | Sequence | EBP |

| | | | | |
|---|---|---|---|---|
| 102 | 1123 | WNB7 3119 | GMEIRPQRHD | 0.04 |
| 103 | 1124 | WNB7 2818 | MEIRPQRHDE | 0.08 |
| 104 | 1125 | WNB7 0591 | EIRPQRHDEK | 1.12 |
| 105 | 1126 | WNB7 2660 | IRPQRHDEKT | 0.1 |
| 106 | 1127 | WNB7 0019 | RPQRHDEKTL | 22.49 |
| 107 | 1128 | WNB7 2661 | PQRHDEKTLV | 0.1 |

This table provides an example of the analysis performed to select candidate B*07 ligands from the WNV genome. The sequence of WNV was parsed into overlapping peptides, 10 amino acids in length, (“10 mers”) overlapping by one amino acid, starting with the first amino acid to be translated from the WNV sequence (AA 0001). The resulting 3,423 “10 mers” were then compared to the EpiMatrix B*07 matrix and evaluated for match to the matrix pattern. Peptides that best matched the matrix received the highest estimated binding probability (“EBP”) which is the value that EpiMatrix uses to describe the probability that the peptide will bind to B*07 in vitro and in vivo. Of the 6 overlapping peptides in this particular region of the WNV sequence shown here, WNVB7 0019 received the best EMX score (22. 49), and could be considered therefore the most likely candidate for in vitro studies (of this set of 6 peptides).

Peptides scoring above an EBP of 20% and below 50% (FIG. 1a) were selected for screening in vitro. Ninety four of the 3,424 10 mers scored above 7%. Of these, 20 were selected, scoring between an EBP of 50 and an EBP of 20. There were 3 peptides scoring above 50 (001, 002, 003, Table 2b); these were not synthesized nor screened for this study because scores in this range are less likely to be ligands and epitopes (TB/HIV Research Lab and EpiVax unpublished results). Two peptides scoring between 50 and 20 (0016 and 0022) were also not tested because they did not fall within a region of the WNV genome belonging to a mature WNV protein, based on information available in the Genbank database (Table 2b). Four peptides could not be synthesized to sufficient purity for this study (0012, 0014, 0021, 0025) and, in addition, the amino acid sequence of peptide 0012 was found to overlap to a significant degree with the human genome (0012) and for that reason was eliminated. Four “non-binder” peptides and a known binder (1291) were also synthesized. A total of 21 peptides were available for testing in vitro.

Of the 3,424 peptides scored in this analysis, 3,330 peptides scored below an EBP of 7% (3,424-94). These were considered unlikely to bind to HLA B*07. Thus, using the EpiMatrix approach, 3330 WNV peptides were set aside as unlikely candidates for HLA B*07 binding studies. This represents a 97% reduction in the complete set of peptides that could have been tested for the WNV NY99 genome (3,330/3,424). Rather than testing every possible peptide in the search for epitopes, some researchers have adopted a standard “overlapping” approach (constructing a set of 10 amino acid long peptides overlapping by 4 amino acids covering the entire genome, for example). This strategy (10/4 OL set) would still require the synthesis of a total of 685 10 mer peptides, approximately 30 times more peptides than were synthesized and tested using the EpiMatrix approach.

### Example 4

### Selection of Peptides

Based on previous analyses, higher EpiMatrix scores suggest greater MHC binding potential. Therefore the top scoring WNV peptides were considered for further evaluation in vitro. Twenty peptides scoring below an EBP of 50 and above an EBP of 20 (Table 3a) were selected for screening in vitro. Peptides scoring with an EBP above 50 were not selected because peptides with this score are less likely to be immunogenic, even though they may bind to B7 in vitro (See, e.g., Jin X, et al., AIDS Res Hum Retroviruses 2000; 16:67-76; De Groot AS, et al., Vaccine. (In press 2001.)). Peptides scoring below an EBP of 20 and above an EBP of 7 are considered likely to bind, however they were not synthesized for the study because of the pilot project (e.g. limited funds) nature of this study.

Four peptides of the lowest scoring WNV peptides (EBP=0.00%, Table 2b) were synthesized to test the hypothesis that low scoring peptides derived from WNV would not bind to HLA B*07 in vitro (predicted non-binders). One well-defined, previously published B*07-restricted epitope (derived from HIV) was also synthesized to serve as a positive control for the assays.

### Example 5

### Cross-Reactivity Analyses

Following the binding analysis, using the proprietary tool Conservatrix, these sequences were aligned and compared for other related flaviviruses and identified (and tagged these peptides in the database) all highly unique sequences in WNV. In an intermediate step designed to avoid selecting epitopes that have any cross-reactivity with ‘self’, each of the highly selected epitopes was passed through the Blast engine at NCBI, using our proprietary tool BlastiMer. Any sequence that was associated with (i.e. is over 80% identical to the 10 amino acid WNV NY99 sequence) a peptide component of equivalent length contained in the human genome (accessible and published to date) was set aside. This only occurred for one of the peptides, peptide 0012, which was found to be highly conserved in the public human genome database at 8 out of the 10 amino acids in the sequence (peptide 0012 was not tested in vitro).

### Example 6

### Peptide Synthesis

Peptides corresponding to the epitope selections were prepared by Fmoc synthesis on an automated Rainen Symphony/Protein Technologies synthesizer. (Synpep, Dublin, Calif.) The peptides are delivered 95% pure as ascertained by HPLC, Mass Spec, and UV scan (insuring purity, mass and spectrum respectively). The peptides were shipped as a lyophilized powder. This powder was diluted in a minimal volume of DMSO and then brought up to stock concentration (1 mg/mi) in RPMI 1640 (Sigma, St Louis, Mo.)). Four peptides could not be purified to specifications, therefore these peptides (0012, 0014, 0021, and 0025) were not evaluated in vitro.

### Example 7

### MHC Binding Studies

The T2B7 binding assay method is well described in the literature and operational in the laboratory. This assay relies on the ability of exogenously added peptides to stabilize the class I MHC/beta 2 microglobulin structure on the surface of transporters associated with antigen processing (TAP)-deficient cell lines. Briefly, HLA B*07 T2 cell lines were prepared by incubating overnight (16 hours) at 26° C. Just prior to the binding assay these cells were washed twice in serum-free media. Solutions of the test peptides at three concentrations (final concentration of 10, 20, and 200 ug/ml in RPMI 1640 (Sigma, St. Louis, Mo.)) were plated in triplicate wells of a 96 well, round-bottom assay plate (Beckton Dickinson, Lincoln Park, N.J.). Sixteen wells containing cells without peptide were included in each plate, serving as the no-peptide control (background control) for the assay.

100,000 cells were added to each well, and the plates were then incubated for four hours at 37° C., 5% CO₂ The plates were then spun at 110×g for 10 minutes at 4° C., supernatant is discarded and the remaining cells are re-suspended. One hundred uL of diluted primary antibody-containing hybridoma supernatant (1:10 dilution of ME1 supernatant produced by HB-119 cell line from ATCC) in staining buffer (PBS, 5% FBS, 0.1% sodium azide) was added to all of the wells except 8 wells per assay plate (each containing 200 ug/ml of one study peptide, these wells served as controls for non-specific binding of secondary antibody). Primary antibody was incubated with the peptide-pulsed cells for 30 minutes at 4° C. After washing three times, the cells were re-suspended, and 100 uL of a 1:250 dilution of FITC-labeled secondary antibody (FITC labeled Goat F(ab′)2 Anti-mouse IgG (H+L) from Caltag) in staining buffer were added to all wells. The plates were incubated for 30 minutes at 4° C., and subsequently washed three times. The contents of each well were then resuspended in 200 uL of fixing buffer (PBS, 1% paraformaldehyde), and aliquoted into FACS tubes.

The 16 negative control wells in each plate contained no peptide but did contain cells, primary antibody and secondary antibody. An additional set of triplicate wells was plated with peptide at the highest concentration (200 ug/ml), but no primary antibody was added to the wells to control for non-specific secondary antibody binding. One positive control peptide (the known B*07 binder) was tested at three concentrations (in triplicate wells) in each assay plate.

Following fixing, the presence of fluorescent secondary antibody on the surface of T2 cells (gated to the appropriate cell size) was measured at 488 nm on a FACScan flow cytometer (Becton-Dickinson, New Jersey). The mean linear fluorescence (MIS) of 10,000 events was measured and compared to the background fluorescence of cells plated in (no peptide) control wells. A positive response was defined as a 10% increase over baseline MLF and p<0.05, in more than one concentration in each of four assays. The assays are repeated 4 times for each concentration of peptide. Each peptide was tested in a total of 36 wells (triplicate wells, three concentrations, four assays).

The B*07 molecule was considered to be stabilized on the surface of the T2B7 cells if the average of the mean linear fluorescence for the triplicate wells at each concentration of peptide was >10% higher than the average of the 16 negative control wells (and p<0.05 in two-way comparison by ANOVA). Binding was rated as strong, moderate, weak, or none, based on the number of significantly positive wells by pair-wise ANOVA. See Table 2, supra.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

1. A vaccine comprising:
one or more West Nile Virus (WNV) vaccine candidate peptides selected from the group consisting of SEQ ID NO: 1-95, in an immunologically acceptable excipient.

2. The vaccine of claim 1, wherein the peptide is between 8 amino acids and 10 amino acids in length.

3. The vaccine of claim 1, wherein one or more of the WNV vaccine candidate peptides has an amino acid sequence selected from the group consisting of SEQ ID NOs: 5, 8, 9, 13, 15, and 17-20.

4. The vaccine of claim 1, wherein the peptide is complexed to a carrier protein.

5. The vaccine of claim 1, wherein the peptide is a recombinant fusion protein.

6. The vaccine of claim 1, wherein the excipient is an adjuvant.

7. A recombinant WNV vaccine candidate peptide, comprising:
a peptide containing an amino acid sequence selected from the group consisting of SEQ ID NOs: 5, 8, 9, 13, 15, and 17-20, wherein the peptide is expressed from a recombinant polynucleotide.

8. The recombinant peptide of claim 7, wherein the recombinant polynucleotide is a naked DNA vaccine.

9. A method for inducing an anti-WNV immune response, comprising:
administering to a mammalian subject the vaccine according to claim 1.

10. The method of claim 9, wherein the induction of an anti-WNV immune response results in the raising of an anti-WNV antibody.

11. The method of claim 9, wherein the mammalian subject is a human.

12. The method of claim 9, wherein the vaccine is administered orally, topically, parenterally, by viral infection, or intravascularly.

13. A method for inducing an anti-WNV immune response, comprising administering to a mammalian subject the vaccine candidate peptide according to claim 7.

14. The method of claim 13, wherein the induction of an anti-WNV immune response is the raising of an anti-WNV antibody.

15. The method of claim 13, wherein the mammalian subject is a human.

16. The method of claim 13, wherein the vaccine is administered orally, topically, parenterally, by viral infection, and intravascularly.
